(19) United States
(12) Patent Application Publication
(10) Pub. No.: US 2003/0149090 A1
(43) Pub. Date: Aug. 7, 2003

(54) Compositions for the treatment of infectious diseases

(76) Inventors: Kurt R. Gehlsen, Encinitas, CA (US); Kristoffer Hellstrand, Gotegorg (SE)

Correspondence Address: KNOBBE MARTENS OLSON & BEAR LLP, 2040 MAIN STREET, FOURTEENTH FLOOR, IRVINE, CA, 92614, US

(21) Appl. No.: 10/289,530

(22) Filed: Nov. 5, 2002

Related U.S. Application Data
(60) Provisional application No. 60/338,878, filed on Nov. 6, 2001.

(51) Int. Cl. A61K 31/4172; A61K 31/405

(52) U.S. Cl. 514/400; 514/419

(57) ABSTRACT

Described herein are compositions and methods for the treatment of microbial infection.

### RELATE APPLICATIONS

This application claims priority to U.S. Provisional Patent Application No. 60/338,878, filed on Nov. 6, 2001, which is incorporated by reference in its entirety.

### BACKGROUND OF THE INVENTION

Reactive oxygen metabolites are often produced by the incomplete reduction of oxygen. The complete reduction of one molecule of O₂ to water is a four-electron process. Oxidative metabolism continually generates partially reduced species of oxygen, which are far more reactive, and hence more toxic than O₂ itself. A one-electron reduction of O₂ yields superoxide ion (O₂⁻); reduction by an additional electron yields hydrogen peroxide (H₂O₂), and reduction by a third electron yields a hydroxyl radical (OH.), and a hydroxide ion. Nitrous oxide (NO), is another interesting reactive oxygen metabolite, produced through an alternative pathway. Hydroxyl radicals in particular are extremely reactive and represent the most active mutagen derived from ionizing radiation. All of these species are generated and must be converted to less reactive species if the organism is to survive.

Particular cells of the immune system have harnessed the toxic effects of ROMs as an effector mechanism. Professional phagocytes, polymorphonuclear leukocytes (neutrophils, PMN), monocytes, macrophages, and eosinophils function to protect the host in which they reside from infection by seeking out and destroying invading microbes. These phagocytic cells possess a membrane-bound enzyme system that can be activated to produce toxic oxygen radicals in response to a wide variety of stimuli.

The “increased respiration of phagocytosis” (the respiratory burst) was reported and thought to be a result of increased mitochondrial activity providing additional energy for the processes of phagocytosis. It was later shown that a non-mitochondrial enzymatic system produced the increased levels of oxygen metabolites since the respiratory burst continued even in the presence of mitochondrial inhibitors such as cyanide and antimycin A. In 1968, Paul and Sbarra showed clearly that stimulated phagocytes produced hydrogen peroxide and in 1973 Babior and co-workers established that superoxide was a major product of the oxidase. See Paul and Sbarra, Biochim Biophys Acta 156(1): 168-78 (1968); Babior, et al., J Clin Invest 52(3): 741-4 (1973). It is now generally accepted that the enzyme is membrane bound, exhibits a preference for NADPH (Kₘ=45 μM) over NADH (Kₘ=450 μM), and converts oxygen to its one electron-reduced product, superoxide.

NADPH+H⁺+2O₂→NADP⁺+2H⁺+2O₂⁻

The hydrogen peroxide arises from subsequent dismutation of the superoxide.

2O₂⁻+2H⁺→H₂O₂+O₂⁻

The enzyme activity is almost undetectable in resting (unstimulated) phagocytes, but increases dramatically upon stimulation. Patients with the rare genetic disorder chronic granulomatous disease (CGD), have a severe predisposition to chronic recurrent infection. The neutrophils from these patients engulf foreign matter normally but the respiratory burst is absent and NADPH oxidase activity (and radical production) is undetectable, indicating that the oxidase and its product, the reactive oxygen metabolites, have an important bactericidal function.

Neutrophils and macrophages produce oxidizing agents to break through the protective coats or other factors that protect phagocytosed bacteria. The large quantities of superoxide, hydrogen peroxide, and hydroxyl ions are all lethal to most bacteria, even when found in very small quantities.

While there are beneficial effects of these oxygen metabolites, it is clear that inappropriate production of oxygen metabolites can result in severely deleterious effects. A number of these deleterious effects manifest themselves in the dermal tissues and mucosal membranes of the host. For example, a variety of infections including Helicobacter pylori, Tinea, and Trypanosoma infections can be exacerbated by unwanted concentrations of reactive oxygen metabolites. Effective compositions and methods to reduce and minimize the production and release of ROMs in patients suffering from a variety of disparate disorders would be a great boon to medicine and serve to reduce and eliminate a substantial amount of human suffering.

Topically administered salves, balms and other such medicaments are well known in the art. The application of mud or plant extracts such as aloe vera are just two examples of such medicaments. For a discussion of aloe vera, see U.S. Pat. No. 4,857,328, which is hereby incorporated by reference. The use of two different histamine derivatives as topically administered skin medicaments has also been discussed previously. The first may be found in a series of U.S. patents to Jack et al., which disclose the use of a pharmaceutical composition of water, water soluble vinyl polymer gel, an amine alcohol dispersant and 1H-imidazole-4-ethanamine phosphate to treat certain skin disorders. See U.S. Pat. Nos. 5,294,440; 5,679,337; and 5,716,610. The second is found in U.S. Pat. No. 5,792,784, that discloses a pseudo-dipeptide product obtained by coupling histamine or a methyl-substituted histamine and an amino acid.

### DETAILED DESCRIPTION OF THE PREFERRED EMBODIMENT

The description below relates to compositions and methods for the treatment of microbial infections such as bacterial, protozoan, yeast, fungi, helminth, and other parasitic infections. The described compositions and methods are useful for treating certain disorders caused by a variety of disease etiologies. Exemplary infections include Helicobacter pylori infections, thought to cause ulcers and other gastrointestinal disorders and Streptococcal infections, thought to cause impetigo, erysipelas, cellulitis, necrotizing fascitis, wound infections, streptococcal toxic shock-like syndrome, puerperal fever, rheumatic fever, glomerulonephritis, erythema nodosum, and scarlet fever.

While the actual mechanism of action for the compositions and methods described herein are not well understood, Applicants have hypothesized why the disclosed materials herein are effective in treating such a wide variety of infectious disorders. One theory holds that once an invading organism such as a bacterium, protozoan, yeast, fungus, helminthes, or other parasitic invades a host, the host mounts an immediate inflammatory response to the invader. Inflammation is typically characterized by vasodilation of the local blood vessels, creating excess local blood flow, increased permeability of the capillaries with leakage of large quantities of fluid into the interstitial spaces, and other local and systemic effects. Soon after the onset of inflammation, neutrophils, macrophages, and other cells invade the inflamed area. These cells set about to rid the tissue of infectious or toxic agents. One method these cells use to defend the body from harmful foreign substances includes the production and release of reactive oxygen metabolites.

A variety of reactive oxygen metabolites (ROMS) are produced in the monovalent pathway of oxygen reduction. These ROMs are enzymatically produced by phagocytes such as monocytes and polymorphonuclear neutrophils (PMNs) and frequently released in a respiratory burst. Hydrogen peroxide and other ROMs play an important role in a host's immunological defenses. Nevertheless, ROMs produced in excessive amounts or at inappropriate times or locations can act to damage a host's cells and tissues, and thus can be detrimental to the host.

The effects of ROM production are many faceted. ROMs are known to cause apoptosis in NK cells. ROMs are also known to cause anergy and apoptosis in T-cells. The mechanisms by which ROMs cause these effects are not fully understood. Nevertheless, some commentators believe that ROMs cause cell death by disrupting cellular membranes and by changing the pH of cellular pathways critical for cell survival.

Additionally, phagocytes that undergo a respiratory burst, and produce and release large quantities of ROMs also produce and release secondary cytokines such as tumor necrosis factor-alpha (TNF-α) and interleukin-1 (IL-1). An example of secondary cytokine mediated cell damage is found in the Shwartzman Reaction, where neutrophil mediated cell damage is thought to be activated by TNF and IL-1 (Imamura S, et al., “Involvement of tumor necrosis factor-alpha, interleukin-1 beta, interleukin-8, and interleukin-1 receptor antagonist in acute lung injury caused by local Shwartzman reaction” Pathol Int. 47(1): 16-24 (1997)). This ROM and cytokine release augments the cell damage inflicted by a variety of sources as these potent chemical compounds are disseminated throughout the body. Although released as a defensive measure by the cells of the immune system, the ROMs result in ROM-mediated cell damage and the secondary cytokines cause a rapid deterioration of the patient, resulting often in death.

It is one of the surprising discoveries of the work described herein that compounds that reducing or inhibiting the amount of ROMs produced or released by sources within a subject can facilitate the treatment and recovery of individuals suffering from a variety of microbial infections. Although the underlying etiological causes of these microbial infections may be only be disparately related, the compositions and methods described herein have broad utility in treating them all. On possible explanation for the ubiquitous efficacy of the compositions and methods described herein is that each of the disparate organisms discussed above may share a common feature in that the pathological conditions they cause are exacerbated by enzymatically produced, ROM-mediated oxidative damage, caused by inappropriate and harmful concentrations of ROMs. Regardless of the actual mechanisms by which the compounds and methods described herein function, the administration of compounds that inhibit the production or release of ROMs, or scavenge ROMs, alone or in combination with other beneficial compounds, provides an effective treatment for a variety of microbial infections.

The methods and compounds described herein have utility in treating a variety of microorganism infections. For example, the methods and compounds described herein have utility in treating helminth, fungal, yeast, protozoan, and bacterial infections, including treatment of, for example, Staphlococcal, Steptococcal, Enterohemmorhagic, Clostridium, Neisseria, Helicobacter, Chlamidia, Tinea, Candida, Mycobacterium, and Trypanosoma infections alone or in conjunction with other therapeutic compounds. The compositions and methods described herein also have utility in the treatment of skin disorders such as acne, acne keloidalis nuchae, acne necrotica, acne urticata, actinic keratoses, acute febrile neutrophilic dermatosis, allergic contact dermatitis, alopecia areata, androgenetic alopecia, atopic dermatitis, blue naevus, basal cell carcinoma, boils, bullous emphitigo, candida, chilblains, chloasma, chloracne, chondrodermatitis nodularis, chromoblastomycosis, dermatitis, dermatofibromas, eczema, erythrasma, folliculitis, fungal infections, hand foot and mouth disease, head lice, impetigo, melanoma, plant dermatitis, nail infections, necrobiosis lipoidica, papular urticaria, paronychia, psoriasis, rosacea, scabies, scalp folliculitis, scleroderma, seborrhoca, shingles, tinea, urticaria, and other skin and mucosal conditions or disease states.

The compounds and methods described herein also have utility in the treatment of gastrointestinal, muscle, eye, genitourinary tract, respiratory, blood, liver, kidney, pancreatic, abdominal, throat, stomach, nasopharangeal, and dental disease. These compounds and methods also have utility in promoting incision healing generally, as well as facilitating the healing process in combination with various chemotherapeutic agents traditionally and recently used in treatment for infections caused by helminths, protozoa, fungi, yeast, bacteria, and other human pathogens.

Formulations

The administration of the ROM production or release inhibiting or scavenging compounds can be via an intravenous, intraarterial, rectal, oral, genital, intramuscular, topical route, transdermal, intranodal or respiratory route. To facilitate these routes of administration, a variety of formulations for the application of the described compounds are available. The described formulations facilitate the administration of compounds that inhibit the production or release of reactive oxygen metabolites or scavenge these compounds once released. In one embodiment, the formulations contemplated here comprise a topical vehicle suitable for the administration of an effective amount of the ROM inhibiting and/or scavenging compounds. In another embodiment, the formulations contemplated here comprise a systemic vehicles suitable for the administration of an effective amount of the ROM inhibiting and/or scavenging compounds.

In a preferred example, various histamine or histamine-derived compounds can be used to achieve a beneficial reduction in the concentration of enzymatically produced ROM production and release. The term “histamine” as used herein incorporates a variety of histamine and histamine-related compounds. For example, histamine, the dihydrochloride salt form of histamine (histamine dihydrochloride), histamine diphosphate, other histamine salts, esters, or prodrugs, and H₂ receptor agonists can be included in the definition of histamine. The administration of compounds that induce the release of endogenous histamine from a patient's own tissue stores can also be used to treat microbial infections. For example, such compounds include IL-3 retinoic acid, other retinoids such as 9-cis-retinoic acid and all-transretinoic acid, and allergens. Other ROM production and release inhibitory compounds such as NADPH oxidase inhibitors like diphenlyeneiodonium also have utility in conjunction with the methods described herein. Furthermore, the topical and systemic administration of serotonin and 5HT agonists also have utility in treating microbial infections.

Formulations containing the ROM inhibitory or scavenging compounds described herein are present in concentrations effective to treat microbial disease or infection. When the formulation contains an ROM inhibitory compound, it preferably contains this component in a total concentration of about 0.0001 to about 0.5 percent by weight of formulation, more preferably about 0.001 to about 0.01 percent by weight of formulation, and most preferably about 0.002 to 0.05 percent by weight of formulation.

The compositions and methods described herein further contemplate administrating a variety of ROM scavengers in conjunction with the ROM production and release inhibiting compounds described above. Known scavengers of ROMs include the enzymes catalase, superoxide dismutase (SOD), glutathione peroxidase and ascorbate peroxidase. Additionally, vitamins A, E, and C are known to have scavenger activity. Minerals such as selenium and manganese can also be efficacious in combating ROM-mediated damage. It is intended that the methods described herein include the administration of the compounds listed and those compounds with similar ROM inhibitor activity.

Compounds that scavenge ROMs can be administered in a total concentration of about 0.0001 to about 0.5 percent by weight of formulation, more preferably about 0.001 to about 0.01 percent by weight of formulation, and most preferably about 0.002 to 0.05 percent by weight of formulation. Formulations containing ROM scavengers are administered from 1 to 10 times per day. In each case, the dose and times of application depend on the activity of the administered compound and the causative agent of the infectious disease. The foregoing doses are appropriate for the compounds listed above. Appropriate doses for any particular host can be readily determined by empirical techniques well known to those of ordinary skill in the art.

Nonenzymatic ROM scavengers can be administered in amounts empirically determined by one of ordinary skill in the art. For example, vitamins A and E can be administered in doses from about 1 to 5000 IU per dose, 10 to 500, and 100 to 300 IU. Vitamin C can be administered in doses from 1 μg to 10 gm per dose. Minerals such as selenium and manganese can be administered in amounts from about 1 picogram to 1 milligram per dose. These compounds can also be administered as a protective or preventive treatment for ROM mediated disease states.

The preferred concentration ranges expressed above are generally effective to inhibit the production of or scavenge ROMs already present in the treated area of a subject. Higher concentrations may also be successfully used. Moreover, routine clinical assessments can be used to optimize the concentration at which the compounds described herein are administered. For example, the concentration of histamine can be adjusted to accommodate an infection based upon the causative agent and stage of infection to be treated. Concentrations can also vary based upon the vehicle used as the formulation. A lotion, which is designed to blend into the skin leaving no visible trace might contain a lower concentration of histamine when compared to a cream that is formulated to dry on the skin of the treated subject. Whereas a fluid composition of histamine can be adjusted to accommodate its intravenous administration, alone or in combination with a chemotherapeutic agent, in order to rid the body of the pathogenic microorganism.

The concentration of the ROM inhibiting or scavenging compounds described can vary in accordance with the other ingredients used in the formulation. For example, histamine concentrations can be decreased when compounds that reduce skin irritation are included, such as strontium, aloe vera, chamomile, a-bisabolol, cola nitida extract, green tea extract, tea tree oil, licorice extract, allantoin, urea, caffeine or other xanthines, and glycyrrhizic acid and its derivatives. Likewise, histamine concentrations can be decreased in fluid form by mixture with saline solutions and additives known to those of skill in the art of administered intravenous fluids. These compounds can also be used in the formulation in conjunction with the ROM inhibiting or scavenging compounds discussed above. These compounds can be added to the compositions singularly or in combination with each other. For the use of strontium as a skin anti-irritant see U.S. Pat. No. 5,804,203, hereby incorporated by reference.

In addition, inclusion of various antibiotic, antifungal, antihelminth, and antiprotozoan agents in the compositions described herein can be included in the compositions described herein. Examples of these agents include aminoglycosides, penicillins, antifungals such as amphotericin B, fluoroquinolones, tetracyclines, beta-lactams, sulfonamides and the like. Depending on the recommended routes of administration for these chemotherapeutics, they may either be combined with the compositions described herein, administered concurrently at a separate site, or administered before or after the compositions described herein.

Further, the inclusion of substances such as analgesics are contemplated for inclusion in the described compositions. Also, compounds that result in the stimulation of a host's immune system such as cytokines, (e.g., IL-1, IL-2, IL-12, IL-15, IFN-α, IFN,-β, IFN-γ and the like) can be included in the compositions described herein.

Suitable vehicles and components for use with the formulations of the described herein are well known in the art. Such vehicles include water; organic solvents such as alcohols (such as ethanol); glycols (such as propylene glycol); aliphatic alcohols (such as lanolin); mixtures of water and organic solvents and mixtures of organic solvents such as alcohol and glycerin; lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile); hydrocarbon-based materials such as microsponges and polymer matrices; stabilizing and suspending agents; emulsifying agents; and other vehicle components that are suitable for administration to the skin, as well as mixtures of these components and those otherwise known in the art. The vehicle can further include components adapted to improve the stability or effectiveness of the applied formulation, such as preservatives, antioxidants, skin penetration enhancers and sustained release materials. Examples of such components are described in the following reference works hereby incorporated by reference in its entirety: Martindale—The Extra Pharmacopoeia (Pharmaceutical Press, London 1993) and Martin (ed.), Remington's Pharmaceutical Sciences.

The choice of a suitable vehicle will depend on the particular physical form and mode of delivery that the formulation is to achieve. Examples of suitable forms include liquids (e.g., eye drops, aerosol, insufflation, inhalation, intravenous drip bags, onsite injection syringes, gargles, intramuscular injections, intraparatoneal injections, injection into the spinal fluid of the central nervous system subcutaneous injection, and mouthwashes); solids and semisolids such as gels, foams, pastes (such as capsules, oral administration (including subligual or buccal), pills, implantable devices, biodegradable timed released devices, chews, lozenges, topically applied pastes as well as toothpaste compositions), creams, ointments, “sticks” (such as lipsticks or underarm deodorant sticks), powders and the like; formulations containing microcapsules prepared, for example, by coacervation techniques, or by interfacial polymerization, for example hydroxymethylcellulose or gelatin-microcapsules, respectively, or in colloidal drug delivery systems, for example, liposomes, albumin microspheres, microemulsions, nanoparticles, and nanocapsules, or in macroemulsions; rectal or vaginal suppositories, creams, foams, gels or other ointments; and other forms. An example of toothpastes can be found in U.S. Pat. No. 4,307,076, which discusses toothpaste compositions and is hereby incorporated by reference.

The formulations described herein can be prepared in a variety of physical forms. For example, solids, pastes, creams, lotions, gels, and aqueous liquids are all suitable formulation forms. A difference between these forms is their physical appearance and viscosity, which can be governed by the presence and amount of emulsifiers and viscosity adjusters present in the formulation. Particular topical formulations can often be prepared in a variety of these forms. Solids are generally firm and non-pourable and commonly are formulated as bars or sticks, or in particulate form; solids can be opaque or transparent, and optionally can contain solvents, emulsifiers, moisturizers, emollients, fragrances, dyes/colorants, preservatives, and other active ingredients that increase or enhance the efficacy of the final product. Creams and lotions are often similar to one another, differing mainly in their viscosity; both lotions and creams may be opaque, translucent or clear and often contain emulsifiers, solvents, and viscosity adjusting agents, as well as moisturizers, emollients, fragrances, dyes/colorants, preservatives, and other active ingredients that increase or enhance the efficacy of the final product. Gels can be prepared with a range of viscosities, from thick or high viscosity to thin or low viscosity. These formulations, like those of lotions and creams may also contain solvents, emulsifiers, moisturizers, emollients, fragrances, dyes/colorants, preservatives, and other active ingredients that increase or enhance the efficacy of the final product. Liquids are thinner than creams, lotions, or gels and often do not contain emulsifiers. Liquid products often contain solvents, emulsifiers, moisturizers, emollients, fragrances, dyes/colorants, preservatives, and other active ingredients that increase or enhance the efficacy of the final product.

Suitable emulsifiers for use in the formulations described herein include, but are not limited to ionic emulsifiers; behentirmonium methosulfate, cetearyl alcohol; nonionic emulsifiers like polyoxyethylene oleyl ether, PEG-40 sterate, ceteareth-12, ceteareth-20, ceteareth-30, ceteareth alcohol, PEG-100 stearate, glyceryl stearate, or combinations or mixtures thereof.

Suitable viscosity adjusting agents for use in the formulations described herein include, but are not limited to protective colloids or non-ionic gums such as hydroxyethylcellulose, xanthan gum, magnesium aluminum silicate, silica, microcrystalline wax, beeswax, paraffin, and cetyl palmitate, or combinations or mixtures thereof.

Suitable solvents for use in the formulations of the described herein include, but are not limited to; water, ethanol, butylene glycol, propylene glycol, isopropyl alcohol, isoprene glycol, and glycerin. In addition, combinations or mixtures of these solvents can be used in the formulations described herein.

Suitable surfactants for use in the formulations described herein include, but are not limited to; nonionic, amphoteric, ionic, and anionic surfactants. For example, dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate are contemplated for use with the formulations disclosed herein. In addition, combinations or mixtures of these surfactants can be used in particular embodiments of the disclosed formulations.

Suitable preservatives for use in particular embodiments of the disclosed formulations, but are not limited to; antimicrobials such as methylparaben, propylparaben, sorbic acid, benzoic acid, and formaldehyde, as well as physical stabilizers, and antioxidants such as vitamin E, sodium ascorbate/ascorbic acid, and propyl gallate. In addition, combinations or mixtures of these preservatives can be used in particular embodiments of the disclosed the disclosed formulations.

Suitable moisturizers for use in particular embodiments of the disclosed formulations include, but are not limited to lactic acid and other hydroxy acids and their salts, glycerin, proplyene glycol, and butylene glycol. Suitable emollients include lanolin alcohol, lanolin, lanolin derivatives, cholesterol, petrolatum, isostearyl neopentanoate, and mineral oils. In addition, combinations or mixtures of these moisturizers and emollients can be used in particular embodiments of the disclosed formulations.

Suitable active ingredients in addition to the ROM production and release inhibiting compounds for use in particular embodiments of the disclosed formulations include, but are not limited to alpha hydroxy acids, sunscreens, anti-acne drugs, vitamins and minerals, and various prescription and over-the-counter medications. An example of a sunscreen can be found in U.S. Pat. No. 5,160,731, hereby incorporated by reference. Embodiments of the disclosed formulations also can include of multiple additional active ingredients such as those listed above.

Suitable fragrances and colors for use in particular embodiments of the disclosed formulations include, but are not limited to, FD&C Red No. 40 and FD&C Yellow No. 5. Other examples of fragrances and colors suitable for use in topical products are known in the art.

Other suitable additional ingredients that may be included in particular embodiments of the disclosed formulations include, but are not limited to, abrasives, absorbents, anti-caking agents, anti-foaming agents, anti-static agents, astringents (e.g., witch hazel, alcohol and herbal extracts such as chamomile extract), binders/excipients, buffering agents, chelating agents, film forming agents, conditioning agents, opacifying agents, pH adjusters, and protectants. Examples of each of these ingredients in topical product formulations, can be found in publications by The Cosmetic, Toiletry, and Fragrance Association (CTFA). See, e.g., CTFA Cosmetic Ingredient Handbook, 2^{nd} edition, eds. John A. Wenninger and G. N. McEwen, Jr. (CTFA, 1992).

Also, a variety of product types, including particularly cosmetics, can be formulated in each of the forms described above (i.e., solids, creams, lotions, gels, and liquids). For example, cleansers (such as soaps), shampoos/conditioners, make-up products, and other facial, hand and body products can be formulated in any of the product forms described above: solids, creams, lotions, gels, or liquids. Common solid form products include; suppositories, cosmetics such as lipsticks, pills, capsules, blushes, other makeup products, lozenges, implantation devices, controlled release devices, oral pills, deodorants, and suppositories. Common cream and lotion form products include; urogenital foams, moisturizing products, sunscreens, shampoos/conditioners and other hair care products, as well as other makeup products such as foundations. Common gel products include; oral capsules, anti-acne solutions and skin conditioners. Common liquid form products include; intravenous drip bags, intraarterial drip bags, intramuscular injection, inhalants, aerosols, injection into the spinal fluid, insufflation, ocular drops, nasal sprays, on-site injectable syringes, vapors, soaks, washes, swallows, nail polish (for treatment of Tinea and other fungal nail growth), anti-acne solutions, perfumes/colognes, aftershaves, gargles/mouthwashes, and toners/bracers/skin conditioners.

Other methodologies and materials for preparing formulations in a variety of forms are also described in Anthony L. L. Hunting (ed.), “A Formulary of Cosmetic Preparations (Vol. 2)—Creams, Lotions and Milks,” Micelle Press (England, N.J., 1993). See, for example, Chapter 7, pp. 5-14 (oils and gels); Chapter 8, pp. 15-98 (bases and emulsions); Chapter 9, pp. 101-120 (“all-purpose products”); Chapter 11, pp. 185-208 (foundations, vanishing and day creams); Chapter 12, pp. 209-254 (emollients); Chapter 13, pp. 297-324 (facial treatment products); Chapter 14, pp. 325-380 (hand products); Chapter 15, pp. 381-460 (body and skin creams and lotions); and Chapter 16, pp. 461-484 (baby products); the contents of which are incorporated herein by reference.

The compositions and formulations disclosed herein may also be incorporated into other articles for use. For example, compositions of the described embodiments of the invention may be incorporated into bandages to increase wound healing and reduce subject discomfort. The compositions may be mixed with saline and chemotherapeutic agents in an intravenous drip bag. Methods of incorporating a ROM production and releasing inhibitory compound into a wound dressing are readily apparent to those of ordinary skill in the art. A discussion of incorporating active materials into a wound dressing is found in U.S. Pat. No. 5,116,620, which is hereby incorporated by reference.

Administration of Compound by Injection

Administration of compounds disclosed herein can be through injection. Typical modes of delivery include administration using an intravenous shunt, hypodermic syringe, intravenous drip bag, intramuscular injection, intraparatoneal injection, suppository, inhalation, vapor, transdermal application, infuser, sponges, spraying (including mist, aerosol or foam spraying), dropper application, sprinkling, ointment, soaking, and gargling or rinsing. Other modes of application include applying the compounds and compositions described onto a bandage or wound dressing, or an implantable device, or biodegradable timed release device attached to the infected area, to hold the compounds in communication with a wound site.

Controlled release vehicles can also be used to administer the preferred embodiments of the compounds described herein. The technology and products in this art are variably referred to as controlled release, sustained release, prolonged action, depot, repository, delayed action, retarded release and timed release; the words “controlled release” as used herein is intended to incorporate each of the foregoing technologies.

Numerous controlled release vehicles are known, including biodegradable or bioerodable polymers such as polylactic acid, polyglycolic acid, and regenerated collagen. Known controlled release drug delivery devices include creams, lotions, tablets, capsules, gels, microspheres, and liposomes. Transdermal formulations, from which active ingredients are slowly released are also well known and can be used with a variety of the embodiments described herein.

Controlled release preparations can be achieved by the use of polymers to complex or absorb the histamine. The controlled delivery can be exercised by selecting appropriate macromolecule such as polyesters, polyamino acids, polyvinylpyrrolidone, ethylenevinyl acetate, methylcellulose, carboxymethylcellulose, and protamine sulfate, and the concentration of these macromolecule as well as the methods of incorporation are selected in order to control release of active compound.

Hydrogels, wherein the histamine compound is dissolved in an aqueous constituent to gradually release over time, can be prepared by copolymerization of hydrophilic mono-olefinic monomers such as ethylene glycol methacrylate. Matrix devices, wherein the histamine is dispersed in a matrix of carrier material, can be used. The carrier can be porous, non-porous, solid, semi-solid, permeable, or impermeable. Alternatively, a device comprising a central reservoir of histamine surrounded by a rate controlling membrane can be used to control the release of histamine. Rate controlling membranes include ethylene-vinyl acetate copolymer or butylene terephthalate/polytetramethylene ether terephthalate. Use of silicon rubber depots are also contemplated.

Controlled release oral formulations are also well known. Active compound is incorporated into a soluble or erodible matrix. Hydrophilic gums, such as hydroxymethylcellulose, are commonly used. A lubricating agent such as magnesium stearate, stearic acid, or calcium stearate can be used to aid in the tableting process.

In a preferred embodiment, the method of administration can be either local or systemic injection or infusion. Other methods of administration are also suitable. The compounds can be administered intraperitoneally or in another parenteral method. Solutions of the active compounds in the form of free acids or pharmaceutically acceptable salts can be administered in water with or without a tenside such as hydroxypropylcellulose. Dispersions making use of glycerol, liquid polyethyleneglycols, or mixtures thereof with oils can be used. Antimicrobial compounds can also be added to the preparation.

Injectable preparations may include sterile water-based solutions or dispersions and powders that can be dissolved or suspended in a sterile medium prior to use. Carriers such as solvents or dispersants containing, e.g., water, ethanolpolyols, vegetable oils and the like can also be added. Coatings such as lecithin and tensides can be used to maintain suitable fluidity of the preparation. Isotonic substances such as sugar or sodium chloride can also be added, as well as products intended to retard absorption of the active ingredients, such as aluminum monostearate and gelatin. Sterile injectable solutions are prepared in the familiar way and filtered before storage and/or administration. Sterile powders can be vacuum-dried or freeze-dried from a solution or suspension.

Nebulizer therapy, vaporizers, or inhalers may be used to administer the preparation. A fine liquid mist of the preparation, alone or in addition to chemotherapeutic drugs specific to the infection can be administered to treat respiratory infections.

Eye drop and ointments can be used to administer the preparation of the described embodiment of the invention. The preparation can be delivered by drop either alone or mixed with additional chemotherapeutics specific to the infection. Ointments containing the preparation of the described embodimentwith or without an antibacterial, antiprotozoan, antihelminth, or antifungal agent are administered to the eye for prolonged exposure as for example while sleeping.

Surgical implants are devised which contain the preparation herein described. For example dental implants that time release the compound of the described embodimentmay be used to reduce inflammation of the gums due to tooth decay. In addition, the composition herein described is mixed with a antibiotic or antiseptic that inhibits the growth of Streptococcus mutans in the oral cavity. The surgical device may be implanted along the gums near the focus of tooth decay or attached externally to a tooth.

Suppositories and enemas that contain the preparation herein described are planted in the infected orifice in order to reduce inflammation from the body's response to the infection. The preparation may be delivered by suppository alone or in combination with other chemotherapeutics. An enema is appropriate for delivery of the preparation alone, or in addition to other chemotherapeutics, for microbial infections positioned higher-up in the human gastrointestinal tract.

Intravenous administration of the preparation herein described is delivered by syringe into the blood stream, muscle, peritoneum cavity, an individually infected organ or system of organs, bone, lymph cavities, spinal cavity, sinus cavity, or the like either alone or in combination with other chemotherapeutics specific to the infection intended for treatment.

In another embodiment, transdermal patches, steady state reservoirs sandwiched between an impervious backing and a membrane face, and transdermal formulations, can also be used to deliver histamine and histamine agonists. Transdermal administration systems are well known in the art. Occlusive transdermal patches for the administration of an active agent to the skin or mucosa are described in U.S. Pat. Nos. 4,573,996, 4,597,961 and 4,839,174, which are hereby incorporated by reference. One type of transdermal patch is a polymer matrix in which the active agent is dissolved in a polymer matrix through which the active ingredient diffuses to the skin. Such transdermal patches are disclosed in U.S. Pat. Nos. 4,839,174, 4,908,213 and 4,943,435, which are hereby incorporated by reference.

Present transdermal patch systems are designed to deliver smaller doses over longer periods of time, up to days and weeks, whereas the embodiment described herein would specifically deliver an effective dose of histamine in a range of between about 1 and 60 minutes, depending upon the dose, with a preferred dose being delivered within about 5 minutes. These patches allow rapid and controlled delivery of histamine. The size of these patches are adapted for placement directly over a wart, a lesion, a herpetic wound, infection site, or the like. A rate-controlling outer microporous membrane, or micropockets of histamine dispersed throughout a silicone polymer matrix, can be used to control the release rate. Such rate-controlling means are described in U.S. Pat. No. 5,676,969, which is hereby incorporated by reference. In another preferred embodiment, the histamine is released from the patch into the skin of the patient in about 30 minutes or less. In a preferred embodiment, the histamine is released from the patch at a rate of between about 0.025 mg to 0.3 mg per minute for a dose of between about 0.2 mg and 3 mg per patch.

These transdermal patches and formulations can be used with or without use of a penetration enhancer such as dimethylsulfoxide (DMSO), combinations of sucrose fatty acid esters with a sulfoxide or phosphoric oxide, or eugenol. The use of electrolytic transdermal patches is also within the scope of the described embodiment. Electrolytic transdermal patches are described in U.S. Pat. Nos. 5,474,527, 5,336,168, and 5,328,454, the entire contents of which are hereby incorporated by reference.

In another embodiment transmucosal patches designed for placement over a wound, lesion, or wart, abrasion, blemish, or infection site can be used to administer the active ingredients of the described embodiment. An example of such a patch is found in U.S. Pat. No. 5,122,127, which is hereby incorporated by reference. The described patch comprises a housing capable of enclosing a quantity of therapeutic agent where the housing is capable of adhering to mucosal tissues, for example, in the mouth. A drug surface area of the device is present for contacting the mucosal tissues of the host. The device is designed to deliver the drug in proportion to the size of the drug/mucosa interface area. Accordingly, drug delivery rates may be adjusted by altering the size of the contact area.

The housing is preferably constructed of a material that is nontoxic, chemically stable, and non-reactive with the compounds of the embodiment described herein. Suitable construction materials include: polyethylene, polyolefins, polyamides, polycarbonates, vinyl polymers, and other similar materials known in the art. The housing can contain means for maintaining the housing positioned against the mucosal membrane. The housing can contain a steady state reservoir positioned to be in fluid contact with mucosal tissue.

Steady state reservoirs for use with the compounds of the described embodiment will delivery a suitable dose of those compounds over a predetermined period of time. Compositions and methods of manufacturing compositions capable of absorption through the mucosal tissues are taught in U.S. Pat. No. 5,288,497, which is hereby incorporated by reference. One of skill in the art could readily how to include the compounds of the described embodiment in these and related compositions.

The steady state reservoirs for use with the described embodiment are composed of compounds known in the art to control the rate of drug release. In one embodiment, the transmucosal patch delivers a dose of histamine over a period of time from about 2 to 60 minutes. The steady state reservoir contained within the housing can carry doses of histamine and other ROM production and release inhibitory compounds in doses from about 0.2 to 5 mg per patch. Transdermal patches that can be worn for several days and that release the compounds of the described embodiment over that period of time are also contemplated. The reservoirs can also contain permeation or penetration enhancers, as discussed above, to improve the permeability of the active ingredients of the described embodiment across the mucosal tissue.

Another method to control the release of histamine incorporates the histamine into particles of a polymeric material such as polyesters, polyamino acids, hydrogels, poly lactic acid, or ethylene vinylacetate copolymers.

Alternatively, instead of incorporating histamine into these polymeric particles, histamine is entrapped in microcapsules prepared, for example, by coacervation techniques, or by interfacial polymerization, for example hydroxymethylcellulose or gelatin-microcapsules, respectively, or in colloidal drug delivery systems, for example, liposomes, albumin microspheres, microemulsions, nanoparticles, and nanocapsules, or in macroemulsions. Such technology is well known to those of ordinary skill in pharmaceutical sciences.

In another embodiment, histamine, a H₂-receptor agonist, in a total concentration of about 0.0001 to about 0.5 percent by weight of formulation, more preferably about 0.001 to about 0.01 percent by weight of formulation, and most preferably about 0.002 to 0.05 percent by weight of formulation can be administered. ROM scavenging compounds can also be administered in combination with the ROM production and release inhibitory compounds described above.

Administration of each dose of histamine can occur from once a day to up to about twenty times a day, with five times a day being preferred. Additionally, the compounds, compositions and formulations of the described embodimentcan be administered quantum sufficiat, or as much as may be needed to ease the pain or discomfort of the subject. Hourly administrations are also contemplated, however, administrations should not exceed 20 per day.

The administration of the compounds of the described embodiment can be alone, or in combination with other compounds effective at treating the various medical conditions contemplated by the embodiment described herein. For example, histamine can be used to treat a patient suffering from a Klebsiella infection in conjunction with other compounds such as cefazolin to ease subject discomfort while ridding the body of the infectious agent. Further, the compounds of the described embodiment can be used with a variety of antibacterial, antifungal, antiprotozoan, and antihelminth compounds known and administered by those of skill in the art. Also, the compounds of the described embodiment, such as histamine, can be administered with a variety of analgesics, anesthetics, or anxiolytics to increase patient comfort during treatment.

Administration of each dose of a compound which induces histamine release can occur from once per day to up to about ten times a day, with five times per day being preferred. Alternatively, administration can be as often as the subject requires to ease infection site, wound, or skin lesion discomfort. Administration is contemplated as being injectable, oral, inhalable, suppository, or topical and can incorporate a controlled release mechanism of the type disclosed above. Any controlled release vehicle capable of administering a therapeutically effective amount of a compound that induces the release of endogenous histamine stores can be used.

Administration of ROM production and release inhibitory compounds by injection in conjunction with the topical administration of these compounds is also contemplated. The administration of these compounds is taught in the co-pending application entitled, “Treatment and Prevention of Reactive Oxygen Metabolite-Mediated Cellular Damage,” which is hereby incorporated by reference.

It is one of the surprising discoveries of the described embodiment of the invention that compounds that reduce the amount of ROMs produced or released by sources within a subject can facilitate the treatment and recovery of individuals suffering from a variety of medical conditions. The conditions contemplated as treatable under the described embodiment result from a disparate number of etiological causes. Nevertheless, they share a common feature in that their pathological conditions are either caused or exacerbated by enzymatically produced, ROM-mediated oxidative damage, caused by inappropriate and harmful concentrations of ROMs. Thus, the administration of compounds that inhibit the production or release of ROMs, or scavenge ROMs, alone or in combination with other beneficial compounds, provides an effective treatment for a variety of medical conditions.

The described embodiment of the invention contemplates compounds and methods that are efficacious in treating a variety of medical conditions wherein ROMs play an active, detrimental role in the pathological state of the disease.

All substances added to the preparation must be pharmaceutically acceptable and essentially nontoxic in the quantities used. The preparation and formulations that produce a delayed release are also part of the invention.

The preparation is supplied in dosage units for a uniform dosage and to facilitate administration. Each dosage unit contains a predetermined quantity of active components to produce the desired therapeutic effect, along with the requisite quantity of pharmaceutical carriers.

Although it is stated in the examples that the administration was given in a single dose, it is obvious that the compounds can be distributed over longer periods of time for treatment of bacterial, fungal, protozoan, helminth, or other parasitic infections which cause inflammation.

The daily dose can be administered as a single dose or it can be divided into several doses, should negative effects occur.

Such conditions include but are not limited to: bacterial infections, fungal or yeast infections, protozoan infections, amoeba infections, and helminth infections. Many of the species listed below are already, or are becoming, resistant to contemporary chemotherapeutic treatments. Thus, when considering the following, please note that the causative agent of any particular infection should be analyzed individually for chemotherapeutic susceptibility in order to render optimal treatment to the patient. Furthermore, each microbial infection may be susceptible to several classes of chemotherapy compounds.

Pneumocystis carinii infections are treated with atovaquone suspension or dapsone with trimethoprim or pentamidine. See Antimicrobial Use Guidelines; University of Wisconsin Hospital 8^{th} Edition June 1996. Clindamycin is used to treat Bacteroides fragilis and Staphlococcus aureus infections as well as aerobic Gram negative bacilli infections. See Young and Mangum, NeoFax, 8th Edition, 1995, page 18. Metronidazole is used to treat B. fragilis, bacterial vaginosis, trichomonal vaginitis, Giardiasis, Clostridium difficile colitis, Entamoeba histolytica, and H. pylori infections. See Rollo I M: Miscellaneous drugs used in the treatment of protozoal infections. In: Gilman A G et al, The Pharmacological Basis of Therapeutics 6th ed, MacMillan Publ, New York, 1980. p. 1077. Additionally, Helicobacter pylori is commonly treated with amoxycillin, clarithromycin, tetracycline, and metronidazole. See Physicians' Desk Reference 58ed. 2002, Medical Economics/Thomson Healthcare p.1471-1474, 403-411, 2893, and 1405-1406 (hereinafter “PDR 2002”). Mycobacterium leprae and M. avium are treated with clofazimine. See National Institutes of Health (NIH), Warren Grant Magnuson Clinical Center (CC) Pharmacy Department, Bethesda, Md. 20892.

Helicobacter pylori causes chronic, often life-long gastritis in humans. A general feature of the host immune response to H. pylori infection is a dense infiltration of the sub-epithelial gastric lamina propria by phagocytes, mainly monocyte/macrophages and neutrophilic granulocytes, and lymphocytes, including those mediating protection against infection such as natural killer (NK) cells and T cells.

Nitrofurantoin is used to treat urinary tract infections. See PDR 2002, p.2891-2892. Dientamoeba fragilis and Cryptosporidial diarrhea are treated with paromomycin. See Clin Infect Dis 1992;15:726; Am J Med 1996;100:370. Neisseria gonorrhoeae is treated with spectinomycin. See U.S. National Library of Medicine, 8600 Rockville Pike, Bethesda, Md. 20894. Escherichia coli, Proteus mirabilis, Klebsiella pneumonia, and Enterobacter species are generally treated with trimethoprim. See PDR 2002, p.2265-2267. Vancomycin is still used to treat methicillin-resistant Staphylococcus aureus, Clostridium difficile, and Corynebacterium infections. See PDR 2002, p.1970-1978.

The following types of infections have been treated with the Beta-lactams family of chemotherapeutics. Aztreonam is used to treat serious infections with aerobic Gram-negative bacilli. See PDR 2002, p.1276-1279. Cefinetazole is used to treat soft tissue infections, bone infections, and infections caused by penetrating abdominal trauma. See Antimicrobial Use Guidelines, Eighth Edition, University of Wisconsin Hospital, June 1996. Loracarbef is used to treat acute otitis media or sinusitis. See PDR 2002, p.2251-2254. Imipenem and Cilastatin are used to treat Pseudomonas aeruginosa, Enterobacter, Serratia, or Citrobacter infections. See PDR 2002, p.2158-2164.

The following penicillins have been found useful in treating the following infections. Amoxicillin is used to treat acute otitis media, bacterial endocarditis prophylaxis, and enterococcal infections. See PDR 2002, p.1471-1474. Ampicillin is used to treat Escherichia coli, Proteus mirabilis, enterococcal endocarditis, neonatal meningitis, Listeria meningitis/sepsis, Haemophilus influenzae, miningitis, shigellosis, salmonellosis, or typhoid fever. See Antimicrobial Use Guidelines, 8th Edition, University of Wisconsin Hospital, June 1996. Amoxicillin and clavulanate are used to treat otitis media and acute sinusitis. See PDR 2002, p.1471-1474 and 1482-1490. Dicloxacillin is used to treat infections caused by penicillinase-resistant, methicillin-susceptible staphylococci. See Olin B R. Systemic Anti-infectives, Antibiotics, Penicillins. In Facts and Comparisons Drug Information. St. Louis, Mo.: Facts and Comparisons, 1993, 1686-732. Penicillin G is used to treat infections including pneumococci, beta-hemolytic streptococci, viridans streptococci, meningococci, Clostridia, susceptible Staphylococci and Pasteurella multocida, neurosyphilis, actinomycosis, anthrax, Micrococcus infections, and spirochete infections (Lyme disease and syphilis). See PDR 2002, p. 2240-2243. Penicillin V K is used to treat streptococcal pharyngitis, streptococcal pharyngitis, streptococcal otitis media, and sinusitis. See Antimicrobial Use Guidelines, 8th Edition, University of Wisconsin Hospital, June 1996. Nafcillin is used to treat Staphylococcus aureus and mixed Gram positive infections. See McCracken and Nelson, Antimicrobial Therapy for Newborns, 2nd Edition, 1983. Benitz & Tatro, Pediatric Drug Handbook, p. 546, 1988. Young & Mangum, NeoFax, p. 34, 1993. Pseudomonas aeruginosa infections are treated with ticarcillin. Id. at p. 543. Ampicillin and sulbactam are used to treat Haemophilus influenzae infections, human or animal bite wounds, urinary infections, and soft tissue infections such as diabetic foot ulcers. Id. at p. 535.

Prophylaxis and treatment of malaria due to Plasmodium vivax, P. malariae, P. ovale, and susceptible strains of P. falciparum is typically through chloroquine phosphate. The family of drugs called sulfonamides is used to treat the following infections. Chloroquine-resistant P. falciparum is treated with sulfadiazine, quinine, and pyrimethamine. See Goldsmith, R. S., Antiprotozoal Drugs in Basic and Clinical Pharmacology (Katzung, B. G., ed) Appleton-Lange, 1998, pp. 838-861. The combination of trimethoprim and sulfamethoxazole is used to treat urinary tract infections, acute prostatitis, P. carinii, shigellosis, typhoid fever, enteropathogenic Escherichia coli, travelers diarrhea, Stenotrophomonas maltophilia, Burkholderia cepacia, methicillin-resistant Staphylococcus aureus, and a typical mycobacterial infections. Id.

Methicillan-resistant Staphlococcus aureus (MRSA) are staphylococci that are resistant to methicillin and other commonly used antibiotics and they have a unique gene that produces resistance. Therefore, alternate antibiotics must be used to treat MRSA. Vancomycin has been the most effective and reliable drug in these cases. See Koren et al, J Peds, V110 N5, p. 797, May 1987. Benitz & Tatro, Pediatric Drug Handbook, p. 571, 1988. Leonard et al, Ped Inf Disease J, V8 N5, p. 282, May 1989. Yeh, Neonatal Therapeutics, 2nd Ed, p.198,1991.

The following microbial infections have been found susceptible to the Family of chemotherapeutics called tetracyclines. See Ziv & Sulman, Am. J. Vet. Res. 35:1197, 1974. USPDI, 11th edition, 1991 USPDI, 15th edition, 1995 BM6th88, Huber, W. G., Tetracyclines, in Veterinary Pharmacology and Therapeutics, 6th edition, eds. Booth, N. H. and McDonald, L. E., Iowa State University Press, 1988. Rang, H. P. and M. M. Dale. Pharmacology, Churchill Livingstone, New York 1987, Chapter 30. Bowersock, T., 1995. Personal communication. Mycoplasma pneumoniae, Chlamydia trachomatis, brucellosis, bartonellosis, rickettsial infections, Lyme disease, mefloquine resistant malaria, chronic bronchitis, Balantidium coli, Pseudomonas pseudomallei, Pseudomonas mallei, and Helicobacter pylori can be treated with Doxycycline or Tetracycline. Id. Minocycline is used to treat Neisseria meningitidis and Mycobacterium marinur. Id. Tetracycline is used to treat Mycoplasma pneumoniae and Chlamidia trachomatis. Id.

Cefazolin is used to treat Klebsiella or Escherichia coli pneumonia or wound infections. See Harriet Lane Handbook, p. 619, 2000. CHLA Pediatric Dosing Handbook and Formulary, p.182, 1999. Neonatal Medications and Nutrition, p. 90, 1999. Cefixime is used to treat penicillin resistant strains of Gonorrhea, acute sinusitis, and acute otitis media. See Girgis N I, Kilpatrick M E, Farid Z, et al: Cefixime in the treatment of enteric fever in children. Drugs Exp Clin Res 1993; 19:47-49; Johnson C E, Carlin S A, Super D M, et al: Cefixime compared with amoxicillin for treatment of acute otitis media. J Pediatr 1991; 119:117-122. Cefpodoxime proxetil is used to treat sinusitis. See PDR 2002, p. 28602864. Pseudomonas aeruginosa is treated with ceftazidime. Id. at 1499-1502. Streptococcus pneumoniae, Haemophilus (Branhamella) influenzae, Moraxella catarrhalis, staphylococci and streptococci skin infections, acute prostatitis caused by E. coli, P. mirabilis, and Klebsiella, are treated with cephalexin. Id. at 1237-1238.

The family of drugs called fluoroquinolones has been used to treat the following infections. Neisseria meningitidis, Pseudomonas aeruginosa, severe enteric infections with Salmonella, Shigella, Campylobacter, or enteropathogenic Escherichia coli, and Gram-negative osteomyelitis are treated with ciprofloxacin. See PDR 2002, p. 893-903. Neisseria gonorrhoeae, non-febrile traveler's diarrhea, chronic prostatitis are included among those infections treated with norfloxacin. Id. at 2051-2053.

Toxoplasma gondii is the causative agent of toxoplasmosis and is treated with pyrimethamine and sulfadiazine. See PDR 2002, p. 1511-1512 and CDC. Availability of sulfadiazine—United States. MMWR 1992;41:950-1.

Mycobacterium avium complex, penicillin-resistant Streptococcus pneumoniae are treated with clarithromycin. See PDR 2002, p. 403-411. Chlamydia trachomatis, Mycoplasma pneumonia, Legionnaire's disease, Chiamydia pneumoniae, Campylobacter jejuni, Bordetella pertussis, Haemophilus ducreyi, acne vulgaris, and Corynebacterium diphtheriae infections are treated with erythromycin. Id. at 455-457.

Aminoglycosides have been used to treat the following microbial infections. Mycobacterium avium complex and resistant tuberculosis is treated with amikacin. See Young and Mangum, NeoFax, 8th Edition, 1995, page 4. Mycobacterium tuberculosis is treated with isoniazid, rifampin, ethambutol, pyrazinamide, and streptomycin. See Core Curriculum on Tuberculosis What the Clinician Should Know 4th. Ed., 2000. Streptomycin is also used to treat streptococcal endocarditis. See U.S. Environmental Protection Agency. 1988. Fact Sheet Number 186 Streptomycin. USEPA. Washington, D.C. Tobramycin is used to treat Pseudomonas aeruginosa. See McCracken and Nelson, Antimicrobial Therapy for Newborns, 2nd Edition, 1983. Benitz & Tatro, Pediatric Drug Handbook, p. 510, 1988. Enterococcal endocarditis is treated with gentamicin and penicillin. See Antimicrobial Use Guidelines, University of Wisconsin Hospital 8^{th} edition, June 1996.

The following antifungals have been found to treat the respective fungal infections. Griseofulvin is used to treat dermatophyte infections (ringworm) of the skin, hair, nails, (Tinea corporis, Tinea barbae, Tinea capitis, Tinea unguium). See E Haneke, I Tausch, M Brautigam et al. Short-duration treatment of fingernail dermatophytosis: a randomized, double-blind study with terbinafine and griseofulvin. See Journal of the American Academy of Dermatology 1995 32:72-7. Miconazole is used to treat Pseudallescheria boydii and systemic Malassezia furfur. See PDR 2002, p. 2661-2662. Amphotericin B is used to treat Ajellomyces capsulatus (i.e. Histoplasma capsulatum), Ajellomyces dermatitidis (i.e. Blastomycoides determatitidis), deep Candida infections, and Coccidioides immitis. are treated with amphotericin B. See Benitz & Tatro, Pediatric Drug Handbook, p. 621, 1988. Medical Letter, Feb. 21, 1992. Candida infections including urogenital and oral infections with Candida albicans are treated with nystatin, amphotericin B, or fluconazole depending on the local of the infection. See Harriet Lane Handbook, p. 161, 1975 Sims, M et al: Prophylactic oral nystatin and fungal infections in very-low-birthweight infants. Am J Perinatology 5(1):33, January 1988. Cryptococcus is treated with amphotericin B or fluconazole. See Cooper C R Jr, McGinnis M R. In vitro susceptibility of clinical yeast isolates to fluconazole and terconazole. Am J Obstet Gynecol 1996;175:162631. Aspergillus infections are treated with amphotericin B or itraconazole. See Drake L A, Dinehart S M, Farmer E R, Goltz R R, Graham G F, et al. Guidelines of care for superficial mycotic infections of the skin: onychomycosis. J Am Acad Dermatol. 1996;34:116-21. Coccidiodes infections are treated with amphotericin B or ketoconazole. See PRD 2002, p.2008-2009.

Generally, the anaerobic species of the genus' Bacteroides, Prevotella, Clostridium, Bifidobacterium, Bilophila, Campylobacter, Centipeda, Escherichia, Eubacterium, Fusobacterium, Gemella, Haemophilus, Lactobacillus, Mobiluncus, Mitsuokella, Neisseria, Peptococcus Peptostreptococcus, Porphyromonas, Propionibacterium, Proteus, Pseudomonas, Sarcina, Selenomonas, Serpula, Staphylococcus, Streptococcus, Veillonella, and Wolinella, and most Gram positive anaerobes are treated with one or a combination of clindamycin, metronidazole, cefriaxone, doxycycline, amoxicillin, or a beta-lactamase inhibitor. See PDR 2002, p. 1405-1406. For most Gram negative anaerobes, treatment is generally accomplished with piperacillin or tobramycin and tazobactam. See Engelkirk, P. et al. Principles and Practice of Clinical Anaerobic Bacteriology, Star Publishing Co. 1992.

For example Streptococcus pyogenes, the causative agent of necrotizing fasciitis (a.k.a. flesh eating bacteria) is treated with one or several of the following: cephalosporin; erythromycin; penicillin; clindamycin; and vancomycin. See Dellinger E P, Severe necrotizing soft-tissue infections. JAMA 1981; 246:1717-1720.

Infection with Absidia is associated with high mortality, particularly in severely ill patients. The fungus usually enters the body through the respiratory tract or is introduced directly onto abraded skin. Primary infection sites are the sinus cavities, lungs, skin, gastrointestinal tract, and central nervous system. Polyenes, primarily amphotericin B, flucytosine, and the azoles are the main antifungals available for prophylaxis and treatment of this fungal infection. See Bennett, J. E. Fungal Infections (Section 15: Infectious Diseases), In Harrison's Principles of Internal Medicine 14th edition, (Isselbacher, K. J., and Braunwald, E., Wilson, J. D., Martin, J. B., Fauci, A. S. and Kasper, D. L., eds) McGraw-Hill, Inc (Health Professions Division), 1998, pp. 1148-1163.

Naegleria fowleri and Acanthamoeba spp. are commonly found in lakes, swimming pools, tap water, and heating and air conditioning units. While only one species of Naegleria is known to infect humans, several species of Acanthamoeba are implicated, including A. culbertsoni, A. polyphaga, A. castellanii, A. astronyxis, A. hatchetti, and A. rhysodes. An additional agent of human disease, Balamuthia mandrillaris, is a related leptomyxid ameba. Acanthamoeba enter the eye via contact lenses or through a corneal cut or sore. Infection or a corneal ulcer results. In addition, Acanthamoeba spp. can cause skin lesions and/or a systemic (whole body) infection. Effective treatment is usually found with topical use of 0.1% propamidine isethionate plus neomycin-polymyxin B-gramicidin ophthalmic solution. See The Pharmaceutical Journal, Vol 264 No 7082 p212-218 (February 2000). Keratoplasty is often necessary in severe infections. Id. Although most cases of brain (CNS) infection with Acanthamoeba have resulted in death, patients have recovered from the infection with proper treatment. Alternatively, new cases of Acanthamoeba respond to sulfonamides while established cases are generally treated with amphotericin B. See De Jonckheere J F: Ecology of Acanthamoeba. Rev Infect Dis 1991 March-April; 13 Suppl 5: S385-7; Martinez A J: Infection of the central nervous system due to Acanthamoeba. Rev Infect Dis 1991 March-April; 13 Suppl 5: S399-402.

Mycoplasma species, two Acholeplasma species and one Ureaplasma species, have been isolated from humans. See Goodman and Gilman (9th Edition), Chapter 49, pp. 1175-1188; 10th Edition, Chapter 49, pp. 1295-1312. Human Pharmacology by Brody, Larner and Minneman (Third Edition), Chapter 55, pp. 735-744. Six of these have the urogenital tract as the primary site of colonization but others, which have the oropharynx and respiratory tract as the primary site, are found occasionally in the urogenital tract because of orogenital contact. Id. Polyene antibiotics are generally used in treatment. Id. The effect, however, must be closely monitored because this drug acts against the cholesterols found in the membrane of mycoplasma, but they can also act against the plasma membrane of the human host cells. Id.

The genus Achromobacter includes the species; anitratus, baumannii, cystinovorum, lwoffi, putrefaciens, xylosoxidans. This is a genus of Gram-negative, aerobic, motile bacteria that occur in water and soil. Some are common inhabitants of the intestinal tract of vertebrates. These bacteria occasionally cause opportunistic infections in humans. They can be treated with fluoroquinolones, piperacillin, or an aminoglycoside in combination with either ceftazidime or pefloxacin. See PDR 2002, p.1499-1502.

In recent years, Acinetobacter species have emerged as clinically important pathogens. Though these organisms are widely prevalent in nature, most human infections are nosocomial. Acinetobacter baumannii is the predominant species. See Hsueh P-R, et al. Pandrug-resistant Acinetobacter baumannii causing nosocomial infections in a university hospital, Taiwan. Emerg Infect Dis (August 2002). Nosocomial A. baumannii infections such as respiratory tract infections, urinary tract infections, meningitis following neurosurgical procedures, and bacteremia mainly affect patients with severe underlying disease in the intensive care unit of a hospital and often, in the setting of a nosocomial outbreak. Combination chemotherapeutic therapy is often used to treat this type of infection. Id.

Endocarditis due to HACEK microorganisms (Haemophilus parainfluenzae, Haemophilus aphrophilus, Actinobacillus actinomycetemcomitans, Cardiobacterium hominis, Eikenella corrodens, and Kingella kingae) can be treated with cefriaxone sodium, ampicillin sodium, and gentamicin sulfates. See Young and Mangum, NeoFax, 5th Edition, 1995, page 14.

Actinomyces, including species such as denticolens, eriksonii, georgiae, gerensceriae, hordeovulneris, howellii, israelii, meyeri, naeslundii, odontolyticus, propionicus, pyogenes, ramosus, slackii, viscosus which cause infections in humans can be treated with minocycline. See Newman, M. G.; Kornman, K.: Antibiotic/Antimicrobial Use in Dental Practice—Chapter 11, 136-147, Quintessence, 1990.

Aerobacter aerogenes, E-coli, Various Proteus, Aerobacter, Klebsiella, Shigella, and Salmonella cause acute and chronic urinary tract infections, cystitis, pyelonephritis, prostatitis, postpartum pyelitis, urethritis, trigonitis. Intestinal infections can also be a result of Salmonella, Shigella, E. coli, and Proteus infections. To treat these Gram negative infections, an effective dose of nalidixic acid (quinolone) is administered to the patient. See Kator, H. and M. Rhodes. 1994. Microbial and chemical indicators. In: Environmental Indicators and Shellfish Safety. C. M. Hackney and M. D. Pierson. (Eds). pp. 30-91. Chapman and Hall Publishers, New York, N.Y.

Aeromonas, including the species; caviae, hydrophila, jandaei, media, salmonicida, schubertii, sobria, trota, veronii can cause wound infections. Antibiotics of choice include aminoglycosides, third-generation cephalosporins, imipenem, meropenem, aztreonam, trimethoprim-sulfamethoxazole (SXT), tetracycline, chloramphenicol, and ciprofloxacin. See Arias, et al. Antimicrobial susceptibility pattern of Gram negative bacteria isolated from enteral feeding Rev Biome. 2000:11;169-174. Gentamicin, SXT, ciprofloxacin, and a third-generation cephalosporin is recommended for wounds containing these microbes. See Altwegg M. 1999. Aeromonas and Plesiomonas. In P R Murray et al. (eds.) Manual of Clinical Microbiology, 7^{th} ed. American Society for Microbiology, Washington D.C.

The preferred treatment for Angiostrongylus cantonensis (eosinophilic meningitis) or Angiostrongylus costaricensis (abdominal angiostrongyliasis) infections is mebendazole. See Department of Pathology, The Johns Hopkins Medical Institutions, Vol. 15 No.12, Microbiology Newsletter, Monday, Mar. 18, 1996. Alternatively, diethylcarbamazine, thiabendazole, and albendazole have been used with “remission” of symptoms. However, surgery is often noted to rid the body of these nematodes. See Barger, I. A. 1992. Control of gastrointestinal nematodes. Haemonchus Workshop, College Park, Md. Actinomyces pyogenes infections are treated with antibiotics and surgical drainage of lesions. Id. In all Actinomyces infections, penicillin is the drug of choice. Actinomyces spp and P. propionicus are generally susceptible to penicillins, the cephalosporins, tetracycline, chloramphenicol, and a variety of other antibiotics. Id.

Arcanobacterium haemolyticum to antimicrobials other than penicillins and erythromycin are fragmentary and based on routine disk diffusion assays and a limited number of strains. McNeil M M, Brown J M. The medically important aerobic actinomycetes: epidemiology and microbiology. Clin Microbiol Rev 1994;7:357-417. A. haemolyticum has been reported to be uniformly susceptible to clindamycin, chloramphenicol, cephalosporins, and fusidic acid.

Ascaris lumbricoides, also known commonly as the “large roundworm” infection and trichuriasis as “whip worm” infection are treatable with piperazine citrate, especially for gastrointestinal or biliary obstruction secondary to ascariasis. This drug causes flaccid paralysis in the helminth by blocking response of the worm muscle to acetylcholine. Mebendazole can also be used for treatment. See Gilles H M: Intestinal nematode infections. In G T Strickland, ed. Hunter's Tropical Medicine. Philadelphia: W B Saunders; 1984: 620 644. This drug causes worm death by selectively and irreversibly blocking uptake of glucose and other nutrients in susceptible adult intestine where helminths dwell. Alternatively, albendazole, mebendazole, and pyrantel pamoate are used to treat ascariasis. See Garcia, L. S. 2001. Diagnostic Medical Parasitology, 4th Ed., ASM Press, Washington, D.C.

Schistosomiasis is caused by digenetic blood trematodes. The three main species infecting humans are Schistosoma haematobium, S. japonicum, and S. mansoni. Safe and effective drugs are available for the treatment of schistosomiasis. See Grove, D. I. and Warren, K. S. Relation of intensity of infection to disease in hamsters with acute schistosomiasis mansoni. American Journal of Tropical Medicine and Hygiene 25: 608 612, 1976. The drug of choice is praziquantel for infections caused by all Schistosoma species. See Befidi Mengue, R. N. et al. (1993). Impact of Schistosoma haematobium infection and of praziquantel treatment on anemia of primary school children in Bertoua, Cameroon. J Trop Med Hyg. 96 (4): 225-30. Oxamniquine has been effective in treating infections caused by S. mansoni in some areas in which praziquantel is less effective. See Brindley P J. Drug resistance to schistosomicides and other anthelmintics of medical significance, Acta Trop 1994;56:213-31.

Blastocystis hominis is a protozoan occasionally found in the intestinal tract of humans, where its pathogenicity is controversial. Infection with this microbe also occurs in other animals. Despite the controversial clinical significance of this organism, metronidazole or iodoquinol has been reported to be effective. See Benitz & Tatro, Pediatric Drug Handbook, p. 650, 1988. Seminars in Pediatric Inf. Diseases, 5(1):15-19, January 1994.

Aspergillus, including species: flavus, fumigatus, glaucus, nidulans, niger, terreus infections in humans can be treated with amphotericin B, itraconazole, granulocyte-macrophage colony-stimulating factor. See Geissmann F et al. Aspergillus brain abscesses: Therapeutic effect of G-CSF and liposomal amphotericin B. Abstract #PB0602, X Int Conf AIDS, Yokohama, 1994. Other investigational therapeutic options for aspergillosis include liposomal amphotericin B and pradimicin. Intranasal and aerosolized amphotericin B may be of prophylactic benefit to reduce nasal carriage in patients with prolonged neutropenia. Id.

Transmission of Babesia occurs through the bite of an infected tick (Ixodes dammini), but transfusion-induced infections are also recognized. See Epidemiologic Notes and Reports Clindamycin and Quinine Treatment for Babesia microti Infections CDC MMWR Weekly Feb. 11, 1983/32(5);65-6,72. A spectrum of infections, ranging from asymptomatic to severe, life-threatening disease with fever, chills, and hemolytic anemia may occur. Treatment of this disease can be accomplished with clindamycin and quinine. Id.

Bacillus, includes the species; alvei anthracis, brevis, cereus, circulans, coagulans, duplex nonliquefaciens, firmus, laterosporus, lentus, licheniformis, macerans, megaterium, mycoides, polymyxa, pumilus, spaericus, stearothermophilys, subtilis, thrungiensis. See Turnbull P C B, Kramer J M, Melling J: Bacillus. p. 187. In Parker M T, Duerden B I (eds): Systematic Bacteriology. Topley and Wilson's Principles of acteriology, Virology and Immunity. Vol. 2. Edward Arnold, Sevenoaks, England, 1990. The clinical forms include (1) cutaneous anthrax (eschar with edema), from handling infected material (this accounts for more than 95 percent of cases); (2) intestinal anthrax, from eating infected meat; and (3) pulmonary anthrax, from inhaling spore-laden dust. Id. Treatment of Bacillus infections is humans is accomplished with non-β-lactam antibiotics for Gram-positive bacteria. Food poisoning is controlled by adequate cooking, avoidance of recontamination of cooked food, and proper storage (efficient refrigeration). Id.

Bacteroides includes the species: amylophilus, asaccharolyticus, bivius, buccae, buccalis, caccae, capillosus, cellulosolvens, corporis, corrodens, denticola, disiens, distasonis, eggerthii, endodontalis, forsythus, fragilis, fragilis, furcosus, galacturonicus, gingivalix, gracilis, hearinolyticus, hypermegas, intermedius, levii, loescheii, macacae, melaninogenicus, merdae, microfusus, multiacidus, nodosus, ochraceus, oralis, oris, oulorum, ovatus, pectinophilus, precutus, ruminocola, salivosus, splanchnicus, stercoris, succinogenes, tectum, termitidis, thetaiotaomicron, uniformis, ureolyticus, veroralis, vulgatus, xylanolyticus, zoogleoformans. While the genus Bacteroides occupies a significant position in the normal flora, they also are opportunistic pathogens, primarily in infections of the peritoneal cavity. See Appelbaum P C, Spangler S K, Jacobs M R: Susceptibilities of 394 Bacteroides fragilis, non-B. fragilis group Bacteroides species, and Fusobacterium species to newer antimicrobial agents. Antimicrob Agents Chemother 1991 June; 35(6): 1214-8. B. fragilis is the most notable pathogen. Id. Antibiotic therapy involving penicillin and clindamycin have been found to be an effective treatment regime in combination with abscess drainage and debridement of necrotic tissue. Id.

Human infections involving Bilophila wadsworthia are best treated with metronidazole, imipenem, chloramphenicol, or combinations of amoxicillin, ticarcillin, ampicillin or piperacillin with β-lactamase inhibitors. Again, most effective treatment of these infections includes surgical drainage of the abscesses and debridement of necrotic tissue. See Brook I: Pediatric anaerobic infection: diagnosis and management. 2nd ed. St Louis, Mo.: Mosby; 1989.

Bordetella includes the species avium, bronchicanis, bronchiseptica, parapertussis, pertussis. Bordetella pertussis, the agent of pertussis (a.k.a. whooping cough), is a very small Gram-negative aerobic coccobacillus. See McCracken and Nelson, Antimicrobial Therapy for Newborns, 2nd Edition, 1983. Benitz & Tatro, Pediatric Drug Handbook, p. 559, 1988. Young and Mangum, NeoFax, 8th Edition, 1995, page 20. Janssens, J et al: “Improvement of Gastric Emptying in Diabetic Gastroparesis by Erythromycin,” NEJM 322(15):1028, Apr. 12, 1990. The antibiotic found effective in treating B. pertussis infections is erythromycin. Additionally, hospitalization and isolation is recommended for seriously ill infants. Id.

Lyme disease is an infection caused by the corkscrew-shaped bacteria Borrelia burgdorferi. This bacteria is transmitted to humans through the bite of deer ticks (Ixodes scapularis) and western black-legged ticks (Ixodes pacificus). Several antibiotics are effective in the treatment of Lyme disease. The present drug of choice is doxycycline, a semisynthetic derivative of tetracycline. Cefuroxime axetil or erythromycin can be used for persons allergic to penicillin or who cannot take tetracyclines. Later stages of Lyme disease, particularly with objective neurologic manifestations, may require treatment with intravenous ceftriaxone or penicillin for 4 weeks or more, depending on disease severity. See Barbour A G. Lyme Disease: The Cause, the Cure, the Controversy. 1996. The Johns Hopkins University Press, Baltimore, Md.

Moraxella catarrhalis (formerly Branhamella) is found only in humans. Presumably, it is spread from person to person. Once someone acquires the bacterium, it usually colonizes the person without causing any immediate symptoms. A symptomatic infection may come later. Treatment of this infection is straightforward. A variety of antibiotics are effective against the organism. See Physicians' Desk Reference. Montvale, N.J.: Medical Economics Co; 2001.

Brucella infections occur primarily through exposure to infected cattle or pigs, but also through drinking unpasteurized milk. Brucellosis is a systemic infection characterized by alternating periods of fever, sweating, and chills. The infection is carried by neutrophils to many body organs. McCracken and Nelson, Antimicrobial Therapy for Newborns, 2nd Edition, 1983. Benitz & Tatro, Pediatric Drug Handbook, p. 559, 1988. Young and Mangum, NeoFax, 8th Edition, 1995, page 20. Janssens, J et al: “Improvement of Gastric Emptying in Diabetic Gastroparesis by Erythromycin,” NEJM 322(15):1028, Apr. 12, 1990. Combination drug therapy, usually including erythromycin, has been found to be effective. Id.

Virtually all persons infected with Campylobacter will recover without any specific treatment. The species of Campylobacter include; butzleri, cinaedi, coli, concisus, cryaerophilus, curvus, fennelliae, fetus, hyointestinalis, jejuni, lari, aridis, mucosalis nitrofigilis, pylori, pyloridis, rectus, sputorum, upsaliensis. Patients should drink plenty of fluids as long as the diarrhea lasts. In more severe cases, antibiotics such as erythromycin or a fluoroquinolone can be used, and can shorten the duration of symptoms if they are given early in the illness. Id.

Cholera can be simply and successfully treated by immediate replacement of the fluid and salts lost through diarrhea. Patients can be treated with oral rehydration solution, a prepackaged mixture of sugar and salts to be mixed with water and drunk in large amounts. This solution is used throughout the world to treat diarrhea. Severe cases also require intravenous fluid replacement. With prompt rehydration, fewer than 1% of cholera patients die. See De S, Choudhuri A, Dutta P, Dutta D, De S P, Pal S C. Doxycycline in the treatment of cholera. Bull WHO 1976;54:177-9.

Most pathologic manifestations of Clonorchis sinensis result from inflammation and intermittent obstruction of the biliary ducts. Praziquantel or albendazole have been found to successfully treat this infection. See Bource P. Successful treatment of Taenia saginata and Hymenolepsis nana by a single oral dose of praziquantel. Journal of the Egyptian Society of Parasitology, 1991, 21(2):303-7.

Q fever is a zoonotic disease caused by Coxiella burnetii. Infection of humans usually occurs by inhalation of these organisms from air that contains airborne barnyard dust contaminated by dried placental material, birth fluids, and excreta of infected herd animals. Humans are often very susceptible to the disease, and very few organisms may be required to cause infection. In general, most patients will recover to good health within several months without any treatment. In serious cases, however, a dose of 100 mg of doxycycline taken orally twice daily for 15-21 days is a frequently prescribed therapy. See Bartlett J G, Dowell S F, Mandell L A, et al: Guidelines from the Infectious Diseases Society of America. Clini Infect Dis. 2000;31. Reprinted with permission of The University of Chicago Press.

The bacterial genus Chlamydia includes the species; pneumoniae, psittaci, and trachomatis, which cause a range of disease from eye, lung, and genitourinary tract infections. Treatment of Chlamydia is accomplished with various antibiotics. Doxycycline is the antibiotic of choice because it is used for extended treatment, can be taken with food, and in inexpensive. However, tetracycline, chloramphenicol, rifampicin, and fluroquinones can also be used. See M R Howell, T C Quinn, C A Gaydos. Screening for Chlamydia trachomatis is asymptomatic women attending family planning clinics. Annals of Internal Medicine 1998 128:277-84.

The genera Escherichia, Klebsiella, Enterobacter, Serratia, and Citrobacter (collectively called the coliform bacilli) and Proteus include overt and opportunistic pathogens responsible for a wide range of infections. Many species are members of the normal intestinal flora. Escherichia coli (E coli) is the most commonly isolated organism in the clinical laboratory. Various antibiotics are the backbone of treatment.

Clostridium include the species: aerotolerans, aldrichii, argentinense, baratii, beijerinckii, bifermentans, botulinum, butyricum, cadaveris, carnis, celerecrescens, cellulofermentans, clostridiiforme, clostridioforme, coccoides, cocleatum, cloinum, cylindrosporum, difficile, disporicum, fervidus, ghoni, glycolicum, haemolyticum, histolyticum, homopropionicum, indolis, innocuum, intestinalis, josui, lentocellum, limosum, litorale, magnum, malenominatum, methylpentosum, novyi, orbiscindens, oxalicum, paraputrificum, perfringens, pfennigii, populeti, proteolyticum, putrificum, ramosum, roseum, scindens, septicum, sordellii, sphenoides, sporogenes, subterminale, symbiosum, tertium, tetani, tetanomorphorum, thermobutyricum, thermocopriae, thermopalmarium, thermopapyrolyticum, and xylanolyticum. Infections caused by this genus range from diarrhea, tetanus, botulism, and gas gangrene. Treatment has been suscessful in many cases due to administration of an effective dose of oral vancomycin, or metronidazole. See Pothoulakis, M. D., et al. Division of Gastroenterology, Beth Israel Deaconess Medical Center, Harvard Medical School, Boston Mass., Participate (Fall 2001).

Symptoms of cryptosporidium include diarrhea, loose or watery stool, stomach cramps, upset stomach, and a slight fever. Some people have no symptoms, yet remain infected as carriers. See Petersen C. Cryptosporidiosis in patients infected with the human immunodeficiency virus. Clin Infect Dis 1992;15:903-9. There is no established specific therapy for human cryptosporidiosis. Rapid loss of fluids because of diarrhea can be managed by fluid replacement and electrolyte balance. Id. Infection in healthy, immunocompetent persons is self-limited, but infection in immunocompromised persons and those in poor health are at higher risk for more severe illness. For persons with AIDS, paromomycin has been used for treatment.

The causal agent for cyclosporiasis has been only recently identified as a unicellular coccidian parasite, Cyclospora cayetanensis. It appears that all human cases are caused by this species. The recommended treatment for cyclosporiasis is a combination of two antibiotics, trimethoprim and sulfamethoxazole, also known as Bactrim, Septra, or Cotrim. Supportive measures include rest, and management of fluid and electrolyte balance. See Remington & Klein, Infectious Diseases of the Fetus & Newborn, p. 559, 1990. Benitz & Tatro, Pediatric Drug Handbook, p. 576-7, 1988.

The cestode Diphyllobothrium latum (the fish or broad tapeworm), the largest human tapeworm. Several other Diphyllobothrium species have been reported to infect humans, but less frequently; they include D. pacificum, D. cordatum, D. ursi, D. dendriticum, D. lanceolatum, D. dalliae, and D. yonagoensis. Treatment of this helminth has been successful using the drug praziquantel. See Bource P. Successful treatment of Taenia saginata and Hymenolepsis nana by a single oral dose of praziquantel. Journal of the Egyptian Society of Parasitology, 1991, 21(2):303-7.

Corynebacterium, includes the species acquaticum, bovis, diphtheriae, equi, haemolyticum, jeikeium, kutscheri, matruchotii, minutissimum, pseudodiphtheriticum, pseudotuberculosis, pyogenes, renale, striatum, ulcerans, ureolyticum, vesiculare, xerosis. C. diphtheriae has been treated with diphtheria antitoxin, to counter the diphtheria toxin, and antibiotics, such as penicillin or erythromycin, to counter the diphtheria bacteria. See PDR 2002, at p.2240-2243.

The Family Enterobacteriaceae (clinically important enterics) include: Citrobacter freundii; Citrobacter diversus; Enterobacter spp.; Enterobacter aerogenes; Enterobacter agglomerans; Enterobacter cloacae; Escherichia coli; Opportunistic Escherichia coli; enterotoxigenic E. coli (ETEC); enteroinvasive E. coli (EIEC); enteropathogenic E. coli (EPEC); enterohemorrhagic E. coli (EHEC); enteroaggregative E. coli (EaggEC); uropathogenic E. coli (UPEC); Klebsiella pneumoniae; Klebsiella oxytoca; Morganella morganii; Proteus mirabilis; Proteus vulgaris; Providencia; Providencia alcalifaciens; Providencia rettgeri; Providencia stuartii; Salmonella enterica; Salmonella typhi; Salmonella paratyphi; Salmonella enteritidis; Salmonella cholerasuis; Salmonella typhimurium; Serratia marcesans; Serratia liquifaciens; Shigella dysenteriae; Shigella flexneri; Shigella boydii; Shigella sonnei; Yersinia enterocolitica; Yersinia pestis; and Yersinia pseudotuberculosis. Infections involving these organisms can often be treated with either aminoglycosides, chloramphenicol, or trimethoprimsulfa-methoxazole. Uncomplicated cases of diarrhea due to Y. enterocolitica usually resolve on their own without antibiotic treatment. However, in more severe or complicated infections, antibiotics such as aminoglycosides, doxycycline, trimethoprim-sulfamethoxazole, or fluoroquinolones may be useful. See Harrison's Principles of Internal Medicine 15th Ed. Chapter 31, (2001).

Ciprofloxacin and fluoroquinolones are the agents of choice for the empiric treatment of invasive and traveler's diarrhea syndromes in the adult patient. They are also the agents of choice when treatment is indicated and the agent is known to be Campylobacter, E. coli (non 0157:H7), Salmonella—non typhoid (although antibiotic treatment may prolong bacterial shedding), Shigella and Yersinia. The antibiotics commonly used for treatment of Shigellosis are ampicillin, trimethoprim/sulfamethoxazole, nalidixic acid, or cyprofloxacin. See Litt J Z, Drug Eruption Reference Manual, New York, Parthenon Publishing (2000).

Salmonella infections usually resolve in 5-7 days and often do not require treatment unless the patient becomes severely dehydrated or the infection spreads from the intestines. Persons with severe diarrhea may require rehydration, often with intravenous fluids. Antibiotics are not usually necessary unless the infection spreads from the intestines, then it can be treated with ampicillin, gentamicin, trimethoprim/sulfamethoxazole, or ciprofloxacin. See PDR 2002, at p. 887-902.

Ehrlichiosis can be a severe illness, especially if untreated, and as many as half of all patients require hospitalization. Severe manifestations of the disease may include prolonged fever, renal failure, disseminated intravascular coagulopathy, meningoencephalitis, adult respiratory distress syndrome, seizures, or coma. The drug used in treatment is often a tetracycline antibiotic, such as doxycycline. See PDR 2002, at p. 2735-2738.

Treatment of Trypanosoma brucei infections should be started as soon as possible and be based on the infected person's symptoms and laboratory results. The drug regiment depends on the infecting species and the stage of infection. Pentamidine isethionate, and suramin (under an investigational New Drug Protocol from the CDC Drug Service) are the drugs of choice to treat the hemolymphatic stage of West and East African Trypanosomiasis, respectively. Melarsoprol is the drug of choice for late disease with central nervous system involvement. See Bryan R, Waskin J, Richards F, et al. African Trypanosomiasis in American travelers: a 20-year review. Travel Medicine. Steffen R, Lobel H O, Haworth J, Bradley D J, eds. Berlin: Springer-Verlag, 1989:384-8.

The protozoan parasite, Trypanosoma cruzi, causes Chagas disease, a zoonotic disease that can be transmitted to humans by blood-sucking reduviid bugs. See Hagar J M, Rahimtoola S H. Chagas' heart disease. Curr Probl Cardiol 1995;20:825-924. Medication for Chagas disease is usually effective when given during the acute stage of infection. Id. The drugs of choice are benznidazole or nifurtimox (under an investigational New Drug Protocol from the CDC Drug Service). See Veloso, V M. et al. Variation in Susceptibility to Benznidazole in Isolates Derived from Trypanosoma cruzi Parental Strains Memorias do Instituto Oswaldo Cruz Vol.96(7): 1005-1011, (October 2001). Once the disease has progressed to later stages, no medication has been proven to be effective. In the chronic stage, treatment involves managing symptoms associated with the disease. Id.

Streptomyces infections require long-term antibiotic treatment and surgical management. See McNeil M M, Brown J M. The medically important aerobic actinomycetes: epidemiology and microbiology. Clin Microbiol Rev 1994;7:357-417. In vitro, S somaliensis is sensitive to rifampicin, erythromycin, tobramycin, fusidic acid, and streptomycin. Strains tested were resistant to trimethoprim. For S. somaliensis infection, treatment with streptomycin and either co-trimoxazole or dapsone is recommended. The average duration of treatment is about 10 months. Id.

Dracunculus medinensis, the guinea worm, is usually treated by careful removal of the worm by winding it on a stick. However, chemotheraputics are also used, such as thiabendazole and metronidazole. See World Health Organization, Fact Sheet No. 98 Dracunculiasis Eradication (March 1998).

The helminth (roundworm nematode) Enterobius vermicularis (previously Oxyuris vermicularis) also called human pinworm, cause infections in humans. Treatment of this infection is carried out through use of a drug called pyrantel pamoate. See RIM Han-Jong; Antihelmintic effect of oxantel pamoate and pyrantel pamoate suspension against intestinal nematode infestations SO:Korean-J-Parasitol 1975 December; 13(2): 97-101.

The trematodes, Fasciola hepatica (the sheep liver fluke) and Fasciola gigantica, are generally parasites of herbivores, but can infect humans accidentally. Unlike infections with other flukes, Fasciola hepatica infections may not respond to praziquantel. The drug of choice is triclabendazole with bithionol as an alternative. See World Health Organization, Fact Sheet No. 191 Triclabendazole and Fascioliasis—A New Drug to Combat and Age Old Disease (April 1998).

The trematode Fasciolopsis buski, is the largest intestinal fluke of humans. Treatment of this infection has been successfully carried out with the drug Praziquantel. See Brown and Neva. Basic Clinical Parasitology (6th ed.), pp 217-261.

Filariasis is caused by nematodes (roundworms) that inhabit the lymphatics and subcutaneous tissues. Eight main species infect humans. Three of these are responsible for most of the morbidity due to filariasis: Wuchereria bancrofti and Brugia malayi cause lymphatic filariasis, and Onchocerca volvulus causes onchocerciasis (river blindness). The other five species are Loa loa, Mansonella perstans, M. streptocerca, M. ozzardi, and Brugia timori. (The last species also causes lymphatic filariasis.) See Hotez, P. J. et al. Emerging and Reemerging Helminthiases and the Public Health of China Emerging Infectious Diseases Vol. 3, No. 3 (July-September 1993); Gubler, D. J. Resurgent Vector-Borne Diseases as a Global Health Problem Emerging Infectious Diseases Vol. 4; No. 3 (July-September 1998). As part of treatment for these infections, antibacterial cream is applied to wounds. Id. Such treatment stops bacterial infections and keeps swelling from worsening. Diethylcarbamazine (under an investigational New Drug Protocol from the CDC Drug Service) and ivermectin are effective for the treatment of filariasis.

Giardia intestinalis, a protozoan flagellate (Diplomonadida) can cause a severe diarrhea infection in humans. Several prescription drugs are available to treat giardiasis, however, metronidazole is the drug of choice. See Hill D R. Giardia lamblia. In: Mandell G L, Bennett J E, Dolin R D, editors. Mandell, Douglas, and Bennett's principles and practice of infectious diseases. 4th ed. New York: Churchill Livingstone Inc.; 1995. p. 2487-91.

The nematode (roundworm) Gnathostoma spinigerum, infects vertebrate animals, including humans. Human gnathostomiasis is due to migrating immature worms. Treatment with albendazole has been successful, as well as concurrent surgical removal. See Garcia L S. Practical Guide to Diagnostic Parasitology. Washington D.C., American Society for Microbiology, 1999. Garcia L S and D A Bruckner. Diagnostic Medical Parasitology. 3^{rd} Edition. Washington D.C., American Society for Microbiology, 1997. The Medical Letter On Drugs and Therapeutics. April 2002. Drugs For Parasitic Infections. Mark Abramowicz (Editor). The Medical Letter, Inc. New Rochelle, N.Y.

Broad-spectrum cephalosporins and fluoroquinolones have been found successful in treating uncomplicated genito-urinary tract infections caused by Neisseria gonorrhea. See The Merck Manual of Diagnosis and Therapy, Sec. 13 Infectious Disease, Ch. 164 Sexually Transmitted Diseases (1995-2000).

Steptococcus pyogenes continues to be exquisitely susceptible to β-lactam antibiotics, and numerous studies have demonstrated the clinical efficacy of penicillin preparations for streptococcal pharyngitis. See Sin, F P. et al. A retrospective review of patients with necrotizing fasciitis presenting to an emergency department in Hong Kong, Hong Kong Journal of Emergency Medicine Vol. 9, No. 1 (January 2002). Similarly, penicillins and cephalosporins have proven efficacy in treating erysipelas, impetigo, and cellulitis, all of which are most frequently caused by S. pyogenes. Id. Group B streptococcal diseases are often treated with penicillin G. Id.

Legionella organisms can be found in many types of water systems. However, the bacteria reproduce to high numbers in warm, stagnant water (90°-105° F.), such as that found in certain plumbing systems and hot water tanks, cooling towers and evaporative condensers of large air-conditioning systems, and whirlpool spas. See Legionella pneumophila infections. In: Pickering L K, ed. Red Book 2000: Report of the Committee on Infectious Diseases. 25th ed. American Academy of Pediatrics; 2000:364-5. Erythromycin is the antibiotic currently recommended for treating persons with Legionnaires' disease. Id. In severe cases, a second drug, rifampin, may be used in addition to erythromycin. Other drugs are available for patients unable to tolerate erythromycin. Id.

Leishmaniasis is a vector-borne disease caused by obligate intracellular protozoa, transmitted by sandflies, of the genus Leishmania. See Boelaert M., et al. Cost-effectiveness of competing diagnostic-therapeutic strategies for visceral leishmaniasis. Bull World Health Organ 1999; 77:667-74. Human infection is caused by about 21 of 30 species that infect mammals. Id. These include a L. donovani complex with 3 species (L. donovani, L. infantum, L. chagasi); a L. mexicana complex with 2 species (L. mexicana and L. amazonensis); L. tropica; L. major; L. aethiopica; and a group of the subgenus Vianna with 4 species (L. (V.) braziliensis, L. (V.) guyanensis, L. (V.) panamensis, and L. (V.) peruviana. Treatment for infections caused by this genus of protozoa is sodium stibogluconate. Id.

Leptospirosis is a bacterial disease that affects humans and animals. It is caused by bacteria of the genus Leptospira. See Radostitis, 0. et al. Verternary Medicine Textbook of the Diseases of Cattle, Sheep, Goats, Pigs and Horses 8th Ed. London, Balliere Tindall, 1994 884-898. In humans it causes a wide range of symptoms, and some infected persons may have no symptoms. Id. Symptoms of leptospirosis include high fever, severe headache, chills, muscle aches, and vomiting, and may include jaundice (yellow skin and eyes), red eyes, abdominal pain, diarrhea, or a rash. If the disease is not treated, the patient could develop kidney damage, meningitis (inflammation of the membrane around the brain and spinal cord), liver failure, and respiratory distress. Leptospirosis is treated with antibiotics, such as doxycycline or penicillin, which should be given early in the course of the disease. Intravenous antibiotics may be required for persons with more severe symptoms. Id.

Pediculus humanus capitis, the head louse, is an insect of the order Anoplura and is an ectoparasite whose only host is humans. See Borror, D. J., C. A. Triplehorn and N. F. Johnson. 1989. An introduction to the study of insects. 6th Ed. Harcourt Brace, New York. p. 875. The louse feeds on blood several times daily and resides close to the scalp to maintain its body temperature. Treatment of this infection is often procured by application of topical medicine called pediculicide, along with physical removal of the louse from the hosts ectoderm. Id.

Listeriosis is mainly a food-borne illness caused by Listeria monocytogenes. People most prone to the disease are pregnant women, newborns, elderly, and those with HIV or other diseases compromising immunity. Ampicillin alone or in combination with gentamicin remains the treatment of choice. See Calder, Jennifer. “Listeria Meningitis in Adults.” Lancet 350 (1997): 307.

The term microsporidia is also used as a general nomenclature for the obligate intracellular protozoan parasites belonging to the phylum microsporidia. See Sandfort J et al. Albendazole treatment in patients with intestinal microsporidiosis. Abstract PO-B10-1491, IX Intl Conf AIDS, Berlin. 1993. To date, more than 1,200 species belonging to 143 genera have been described as parasites infecting a wide range of vertebrate and invertebrate hosts. Id. The treatment of choice for ocular microsporidiosis, caused by Encephalitozoon hellem, E. cuniculi, or Vittaforma corneae is oral albendazole plus topical fumagillin. Id. Albendazole is the drug of choice to treat intestinal infections caused by Enterocytozoon bieneus or Encephalitozoon intestinalis. Id.

Rocky Mountain spotted fever is the most severe and most frequently reported rickettsial illness in the United States. See Archibald L K, Sexton D J: Long-term sequelae of Rocky Mountain spotted fever. Clin Infect Dis 1995 May; 20(5): 1122-5. The disease is caused by Rickettsia rickettsii, a species of bacteria that is spread to humans by Ixodid (hard) ticks. Id. Initial signs and symptoms of the disease include sudden onset of fever, headache, and muscle pain, followed by development of rash. Id. Treatment includes an effective administration of doxycycline. Id.

Trichomonas vaginalis, a flagellate, is the most common pathogenic protozoan of humans in industrialized countries. See Wolner-Hanssen P, Krieger J, Stevens C E, Kiviat N B, Koutsky L, Critchlow C, et al. Clinical manifestations of vaginal trichomoniasis JAMA 1989;261:571-6. Treatment should be implemented under medical supervision, and should include all sexual partners of the infected persons. Id. The drug of choice for treatment is metronidazole. Id. Therapy is usually highly successful. Tinidazole, which is a better-tolerated alternative drug, is not available in the United States. However, strains of Trichomonas vaginalis resistant to both drugs have been reported. Id.

Sporotrichosis is a fungal infection caused by a fungus called Sporothrix schenckii. See Ajello L and R. J. Hay. 1997. Medical Mycology Vol 4 Topley & Wilson's Microbiology and Infectious Infections. 9th Edition, Arnold London. It usually infects the skin. Sporotrichosis is generally treated with potassium iodide, taken by mouth in droplet form. A new drug, called itraconazole, is available for treatment, but experience with this drug is still limited. Treatment is often extended over a number of weeks, until the skin lesions are completely healed. Id.

Syphilis is a complex sexually transmitted disease (STD) caused by the bacterium Treponema pallidum. See Centers for Disease Control and Prevention. 1998 guidelines for the treatment of sexually transmitted diseases. MMWR 47 (RR-1): 1, 1997. It has often been called the great imitator because so many of the signs and symptoms are indistinguishable from those of other diseases. Id. One dose of the antibiotic penicillin will cure a person who has had syphilis for less than a year. More doses are needed to cure someone who has had it for longer than a year. Id. A baby born with the disease needs daily penicillin treatment for 10 days. There are no home remedies or over-the-counter drugs that cure syphilis. Penicillin treatment will kill the syphilis bacterium and prevent further damage, but it will not repair any damage already done. Id.

Toxoplasma gondii is a protozoan parasite that infects most species of warm blooded animals, including humans, causing the disease, toxoplasmosis. See Torres, G. Toxoplasmosis: New Treatment Advances The Gay Men's Health Crisis Newsletter of Experimental AIDS Therapies; Volume 5 Number 3 (Mar. 28, 1991). Treatment is not needed for a healthy person who is not pregnant. Symptoms will usually go away within a few weeks. For pregnant women or persons who have weakened immune systems, pyrimethamine plus sulfadiazine with leucovorin are effective treatment. Id.

Trichinellosis (trichinosis) is caused by nematodes (roundworms) of the genus Trichinella. In addition to the classical agent Trichinella spiralis (found worldwide in many carnivorous and omnivorous animals), four other species of Trichinella are now recognized: T. pseudospiralis (mammals and birds worldwide), T. nativa (Arctic bears), T. nelsoni (African predators and scavengers), and T. britovi (carnivores of Europe and western Asia). See J Dupouy-Camet, W Kociecka, F Bruschi, F Bolas-Fernandez, E Pozio Opinion on the diagnosis and treatment of human trichinellosis Expert Opinion on Pharmacology Vol. 3 (2002). Treatment of these helminth infections involves administration of steroids plus mebendazole. Id.

The nematode (roundworm) Trichuris trichiura causes the human infection known as whipworm. See Cooper, E. S. & Bundy, D. A. P. (1988). Trichuris is not trivial. Parasitology Today. 4(11): 301-306. Treatment involves the administration of the drug mebendazole. Alternatively, albendazole is used as treatment. Id.

Typhoid fever is a life-threatening illness caused by the bacterium Salmonella typhi. See Ryan, Kenneth J. and Stanley Falkow. “Salmonellosis.” In Sherris Medical Microbiology: An Introduction to Infectious Diseases, edited by Kenneth J. Ryan. Norwalk, Conn.: Appleton and Lange, 1994. Three commonly prescribed antibiotics are ampicillin, trimethoprim-sulfamethoxazole, and ciprofloxacin. Id.

Vibrio parahaemolyticus is a bacterium in the same family as those that cause cholera. It lives in brackish saltwater and causes gastrointestinal illness in humans. See World Health Organization Fact Sheet No. 107 Cholera (March 2000). Treatment is not necessary in most cases of V. parahaemolyticus infection. There is no evidence that antibiotic treatment decreases the severity or the length of the illness. Patients should drink plenty of liquids to replace fluids lost through diarrhea. In severe or prolonged illnesses, antibiotics such as tetracycline, ampicillin or ciprofloxicin can be used. Vibrio vulnificus infection is treated with doxycycline or a third-generation cephalosporin (e.g., ceftazidime). Id.

Bacterial vaginosis (BV) is a genito-urinary tract infection caused by various anaerobic bacteria including; Gardnerella vaginalis, Mobiluncus sp., Bacteroides sp. and Mycoplasma hominis. See Ferris D G, Litaker M S, Woodward L, Mathis D, Hendrich J. Treatment of bacterial vaginosis: a comparison of oral metronidazole, metronidazole vaginal gel, and clindamycin vaginal cream. J Fam Pract Vol. 41 (1995). Metronidazole has been found successful to treat this variety of infections. Id.

Leprosy is an infection of the skin, peripheral nerves, and mucous membranes, leading to lesions, hypopigmentation, and loss of sensation (anesthesia), particularly in the cooler areas of the body. See World Health Organization Fact Sheet No. 101 (January 2001). Treatment (including prophylaxis in close contacts) with multi-drug therapy consisting of dapsone, rifampin, and clofazimine is performed on an outpatient basis for 3 to 5 years; vaccination with M bovis BCG has been effective in some endemic areas. Id.

Peptosteptococcus culture and susceptibility studies should be performed to determine the causative organisms and their susceptibility to metronidazole. See Ralph, E. D., and Kirby, W. M. M.: Bioassay of Metronidazole With Either Anaerobic or Aerobic Incubation, J. Infect. Dis. 132:587-591 (November) 1975; or Gulaid, et al.: Determination of Metronidazole and Its Major Metabolites in Biological Fluids by High Pressure Liquid Chromatography, Br. J. Clin. Pharmacol. 6:430-432, 1978.

The following examples teach the methods of the embodiments described herein and the use of the disclosed ROM production and release inhibiting compounds. These examples are illustrative only and are not intended to limit the scope of the described embodiments. The treatment methods described below can be optimized using empirical techniques well known to those of ordinary skill in the art. Moreover, artisans of ordinary skill would be able to use the teachings described in the following examples to practice the full scope of the described embodiments.

### EXAMPLE 1

### Human Monocytes Activated by a Cecropin-Like Peptide Derived from Helicobacter pylori [Hp(2-20)] Triggers Programmed Cell Death (Apoptosis) of Natural Killer Cells and T Cells

Infection with Helicobacter pylori causes chronic gastritis, which is characterized by a dense mucosal infiltration by inflammatory cells such as monocytes/macrophages. Treatment of human monocytes with a cecropin-like H. pylori peptide, Hp(2-20), induces apoptosis of T lymphocytes with CD3ε⁺ phenotype and natural killer cells with CD56⁺ phenotype. Histamine, a gastric mucosal constituent, resuced T cells and NK cells from apoptosis. Histamine may be useful as an adjunct to increase the efficiency of H. pylori-based vaccine protocols.

Helicobacter pylori causes chronic, often life-long gastritis in humans. A general feature of the host immune response to H. pylori infection is a dense infiltration of the sub-epithelial gastric lamina propria by phagocytes, mainly monocyte/macrophages and neutrophilic granulocytes, and lymphocytes, including those mediating protection against infection such as natural killer (NK) cells and T cells. See Agnihotri et al. 1998 Characterization of lymphocytic subsets and cytoking production in gastric biopsy samples from Helicobacter pylori patients. Scand J Gastroenterol. 33:704-9; Li et al. 1999 Reactions from rat gastric mucosa during one year of Helicobacter pylori infection Dig Dis Sci. 44:11624; Takeuchi et al. 2001 Prognostic significance of natural killer cell activity in patients with gastric carcinoma: a multivariate analysis, Am J Gastroenterol. 96:574-8; Ishigami et al. 2000 Prognostic value of intratumoral natural killer cells in gastric carcinoma. Cancer. 88:577-83.

Human monocytes activated by a cecropin-like peptide derived from H. pylori [Hp(2-20)] triggers programmed cell death (apoptosis) of NK cells and T cells. These inhibitory events were mediated by oxygen radicals, induced by Hp(2-20) and produced via the NADPH oxidase activity of monocytes. Histamine dihydrochloride protected NK cells/T cells from the monocyte-induced apoptosis by inhibiting oxygen radical production in monocytes. These effects of histamine were mediated by histamine H2 type receptors. We propose that histamine, analogs thereof with H2 receptor agonist activity, or oxygen radical scavengers/inhibitors may be useful in augmenting the host immune response to H. pylori.

Reagents:

The peptide used, Hp(2-20), AKKVFKRLEKLFSKIQNDK, was synthesized and handled as described in Bylund et al. 2001. Proinflammatory activity of a cecropin-like antibacterial peptide from Helicobacter pylori. Antimicrob Agents Chemother. In press. Histamine dihydrochloride was from Maxim Pharmaceuticals, (San Diego) and ranitidine hydrochloride from Glaxo (Mölndal, Sweden). Superoxide dismutase (SOD), and catalase were purchased from Boehringer-Mannheim, Germany.

Separation of Leukocytes:

Peripheral blood was obtained from healthy blood donors at Sahlgren's Hospital, Göteborg, Sweden. After Ficoll-Hypaque density gradient centrifugation, mononuclear cells were separated into lymphocytes and monocytes using the counter-current centrifugal elutriation (CCE) technique, as described in detail elsewhere and yielded one fraction with >90% monocytes (at a flow rate of 20-22 ml/min) and two lymphocyte fractions, one enriched for CD3ε⁻/56⁺ NK cells (45-50%; at 15-16 ml/min), and one enriched for CD3ε⁺/56⁻ T cells (70-80%; at 13-14 ml/min). See Hansson et al. 1996. Induction of apoptosis in NK cells by monocyte-derived reactive oxygen metabolites. J Immunol. 156:427.

Monocyte Chemotaxis and NADPH-Oxidase Activity:

NADPH-oxidase activity was determined using an isoluminol-enhanced chemiluminescence (CL) system that quantitates extracellular reactive oxygen species (ROS). See Dahlgren, C., Karlsson, A. 1999. Respiratory burst in human neutrophils. J Immunol Methods 232:3-14.

Assays of Apoptosis:

Apoptosis was monitored by use of flow cytometry, as described elsewhere. See Mellqvist et al. 2000. Natural killer cell dysfunction and apoptosis induced by chronic myelogenous leukemia cells: role of reactive oxygen species and regulation by histamine. Blood. 96:1961-8. T cells or NK cells were gated after exposure to monocytes, and the gate was set to comprise lymphocytes with a reduced forward scatter and an increased right angle scatter characteristic of apoptosis. See Hansson et al. 1996. Two additional methods were used to determine apoptosis in NK cells and T cells: analysis of DNA strand breaks by terminal deoxynucleotidyl transferase-mediated bromolated dUTP nick end labelling of DNA fragments (TUNEL assay) and Annexin V staining, as described elsewhere. See Mellqvist et al.; Hansson et al. 1999. Histamine protects T cells and natural killer cells against oxidative stress. J Interferon Cytokine Res. 19:1135-44.

Detection of Lymphocyte Surface and Intracellular Antigens:

One million cells were stained with appropriate fluorescein isothiocyanate (FITC)- and phycoerythrin (PE)-conjugated monoclonal antibodies (Becton & Dickinson, Stockholm, Sweden; 10 μl/10⁶ cells), as described in detail elsewhere. See Hansson et al. 1999. Cells were analyzed by use of flow cytometry on a FACSort with a Lysys II software program (Becton & Dickinson). Lymphocytes were gated on the basis of forward and right angle scatter. The flow rate was adjusted to <200 cells x s⁻¹ and at least 5×10³ cells were analyzed for each sample.

Lymphocyte Apoptosis Induced by Hp(2-20):

Earlier studies have revealed that monocytes/macrophages trigger functional inhibition of NK cells. The finding that Hp(2-20) potently triggers superoxide anion formation in phagocytic cells prompted us to investigate effects of Hp(2-20) on monocyte-NK or T cell interactions. See Bylund et al. 2001. For this purpose, we incubated monocytes at various densities with autologous NK cell enriched lymphocytes and monitored NK cell viability.

Morphological changes characteristic of lymphocyte apoptosis were observed after overnight incubation of lymphocytes with monocytes activated by Hp(2-20). The Hp(2-20)-induced apoptosis was pronounced in NK cells as well as in CD3ε⁺ T cells. Apoptosis in gated lymphocytes was confirmed by DNA fragmentation assay (TUNEL assay) and annexin V staining [not shown] and completely prevented by SOD and catalase (FIG. 1). See Mellqvist et al. 2000; Hansson et al. 1999.

FIG. 1 shows that Hp(2-20) triggers apoptosis in NK cells and T cells. After incubation, cells in a lymphocyte gate were assayed for morphological features of apoptosis (reduced forward and increased right angle scatter) by use of flow cytometry. In A, data are the frequency of apoptotic CD56⁺ (NK; dark gray bars) or CD3ε⁺ (T; open bars) after the following treatments:

lymphocytes incubated in culture medium [control; (1)],

lymphocytes+25% monocytes (2),

lymphocytes+25% monocytes+Hp(2-20) [50 μM; (3)],

lymphocytes+50% monocytes (4),

lymphocytes+50% monocytes+Hp(2-20) (5).

The inset shows apoptosis induced by Hp(2-20)-activated monocytes in all lymphocytes, and these data are the mean±s.e.m. of three separate experiments. The results in B show the apoptosis of lymphocytes induced by 25% monocytes (light gray bars) or 50% monocytes (dark gray bars) activated with Hp(2-20) in cell mixtures treated with SOD+catalase or histamine (50 μM), alone or in the presence of the H2 receptor antagonist ranitidine (50 μM).

Effect of Histamine on NADPH-Oxidase Activity:

Histamine inhibits Hp(2-20)-induced radical production and restores lymphocyte function and viability. Previous studies show that histamine reduces or inhibits NADPH-oxidase dependent formation of oxygen radicals by monocytes and other phagocytic cells. See Mellqvist et al. 2000. The relatively high concentrations of histamine normally present in the gastric mucosa [approximately 10-100 μM] led us to investigate the effects of histamine on Hp(2-20)-induced oxygen radical formation in monocytes. See Bechi et al. 1993. Reflux-related gastric mucosal injury is associated with increased mucosal histamine content in humans. Gastroenterology. 104:1057-63; Lonroth, et al. 1990. Histamine metabolism in human gastric mucosa. Effect of pentagastrin stimulation. Gastroenterology. 98:921-8. Histamine markedly inhibited the oxygen radical formation induced by Hp(2-20), and the specific histamine H2-receptor antagonist ranitidine reversed the inhibition (FIG. 2).

FIG. 2 shows Hp(2-20)-induced oxygen radical production and its inhibition by histamine. Superoxide anion production in elutriated monocytes was investigated by isoluminol-amplified CL (A). Cells were treated with histamine (50 μM) or the histamine H2 receptor antagonist ranitidine (50 μM). Data show mean values±s.e.m. of four separate experiments.

Effect of Histamine on NK Cell and T Cell Function:

Earlier studies show that histamine preserves NK cell and T cell function in the presence of suppressive phagocytes by inhibiting oxygen radical production. See Mellqvist et al. 2000; Hansson et al. 1999. We therefore investigated whether histamine protected NK cells and T cells from the monocyte-dependent, Hp(2-20)-induced apoptosis. Histamine was found to prevent the triggering of NK cell and T cell apoptosis. The histamine-induced protection of T cells and NK cells was antagonized by the H2-receptor antagonist ranitidine (FIG. 3).

FIG. 3 shows Hp(2-20)-induced apoptosis: inhibition by histamine dihydrochloride. Monocytes and/or lymphocytes were prepared as described in Methods. After incubation for 16 hrs, cells in a lymphocyte gate were assayed for morphological features of apoptosis (reduced forward and increased right angle scatter) by use of flow cytometry. Data are the frequency of apoptotic lymphocytes. histamine dihydrochloride, ranitidine, and Hp(2-20) were used at 50 μM, catalase at 100 U/ml, and SOD at 50 U/ml.

The addition of Hp(2-20) to lymphocytes and monocytes, in a mixture aimed at mimicking the mononuclear cell infiltrate of H. pylori-infected gastric tissue, triggered NK cell and T cell death by apoptosis. See Agnihotri et al. 1998; Li et al. 1999. These inhibitory events were prevented by scavengers of NADPH-oxidase-derived oxygen radicals, and were thus by all probability explained by the FPRL1/FPRL2-mediated oxygen radical induction by Hp(2-20). Histamine was found to reduce or inhibit the Hp(2-20)induced formation of oxygen radicals, and thereby to protect T cells and NK cells from apoptotic cell death. This effect of histamine was mediated by histamine H2-receptors expressed by monocytes, and concentrations of histamine similar to those detected in human gastric mucosal tissue were sufficient to mediate the protective effects. See Bechi et al. 1993; Lonroth et al. 1990.

### EXAMPLE 2

### Treatment of Tinea Infection With Histamine Dihydrochloride

The compounds of the described embodiment of the invention are prepared in a cream for topical application according to procedures well known in the art. The ROM production or release inhibition compound histamine dihydrochloride in a concentration of 0.08% by weight of formulation is added to the cream. Two groups of 10 subjects are selected who are suffering from an active Tinea infection. The first group of 10 subjects suffering from fungal infections, the experimental group is treated with the cream containing histamine dihydrochloride. The second group, the control group, is treated with a control cream that is composed of the same ingredients and compounds of the experimental cream, however, it lacks histamine dihydrochloride.

Treatment of the subjects consists of the topical application of the medication four to five times a day at the lesion site. When treating fungal growths, care is taken not to contaminate new areas with fungal spores. Subject in the experimental group experience a decrease in healing time as compared to the control group.

### EXAMPLE 3

### Treatment of Microbial Infections in Conjunction With Other Therapeutic Compounds

The ability of the compositions in the described embodiment to facilitate healing due to microorganisms including, yeast, fungi, bacteria, protozoa, helminth, and amoebic infections using standard compositions is next investigated. The ability of the ROM production and release inhibition compounds of the described embodiment to increase the effectiveness of antimicrobials is evaluated in two groups of 10 subjects each. No subjects are suffering from active infections at the initiation of the study. Group I subjects receive the type of antimicrobial used in treating the specific infection according to the dosage given by the manufacturer. Group II subjects receive the same antimicrobial at the same dose and apply the ROM production and release inhibiting compound histamine dihydrochloride at 0.08% by weight in a form suitable for the type and location of the microbial infection. The healing time of the patient in each group is then monitored. Subjects receive both the antimicrobial and the ROM inhibitory composition demonstrate a faster healing time.

### EXAMPLE 4

### Treatment of Ulcers Caused by Helicobacter pylori Using a Controlled Release Mechanism

A patient diagnosed with ulcers caused by Helicobacter pylori is treated with the compounds of the described embodiment. A controlled release mechanism is replenished with an effective dose of the ROM inhibitory compound NADPH oxidase inhibitor diphenlyeneiodonium and administered to the patient by oral intake of the device. As the device rests in the patient's stomach it simultaneously delivers the compounds of the described embodiment and decomposes in order to allow excretion from the body. The compounds of the described embodiment are administered in combination with chemotherapeutics typically administered for such an infection, such as amoxycillin, clarithromycin, tetracycline, or metronidazole. The administration of the diphenlyeneiodonium is effective in expediting the treatment of gastrointestinal ulcers.

### EXAMPLE 5

### Treatment of Streptococcus pneumonia Lung Infection Using a Nebulizer

A subject diagnosed with Streptococcus pneumonia is treated with compounds of the described embodiment in the form of a nebulizer. The nebulizer is held firmly against the oral-nasal cavities of a patient with a respiratory infection. The nebulizer is used to deliver an aerosol mist as the patient inhales deeply. The aerosol mist contains either solely an effective amount of the preparation herein described or a mixture of the preparation and the chemotherapeutic used often administered for such an infection, such as, cephalexin. The ROM inhibitory compound NADPH oxidase inhibitor diphenlyeneiodonium in a concentration of 0.05% by weight of formulation is delivered as a mist in the patients lungs through a nebulizer. The administration of diphenlyeneiodonium hastens the recovery of a patient with pneumonia.

### EXAMPLE 6

### Treatment of a Chlamidia trachomatis Eye Infection Using Eye Drops and/or an Ointment

A subject presenting an eye infection due to Chlamidia trachomatis is treated with the compositions of the described embodiment using an ophthalmic solution of histamine dihydrochloride at 0.09% by weight of formulation. Commercially available ophthalmic solutions are well known in the art. Additionally, the ophthalmic solution, preferably in drop form, contains erythromycin. Application of the solution to the eye occurs every three hours. A solution containing only the ROM production and release inhibiting compound is given hourly to ease the discomfort of the subject. Application of the solutions reduces the time period of bacterial infection, the damage caused by the bacterial infection, and reduces the discomfort of the patient.

### EXAMPLE 7

### Treatment of an Ascaris Trichuriasis Gastrointestinal Infection Using a Suppository and/or an Enema

A child presenting symptoms of a worm infection is treated with the compounds of the described embodiment. A suppository containing the preparation herein described is delivered to the rectum of a patient with an Ascaris trichuriasis infection (“whip worm”). Gradual dissipation of the preparation positioned on the suppository will decrease inflammation. Additionally, a suppository containing the preparation herein described is administered rectally, in addition to appropriate administration of an antihelminth such as; piperazine citrate, mebendazole, albendazole, or pyrantel pamoate, to a patient infected with Ascaris trichuriasis in order to kill the worm(s) and decrease inflammation. Likewise, an enema is used to deliver the above-described composition in addition to the antihelminth for the same purposes of elimination of the helminth and reduction of inflammation for helminths that position themselves higher-up in the human gastrointestinal tract, such as an Ascaris lumbricoides (round worm) infection. Renewed suppositories and repeat enemas are administered in addition to the other chemotherapeutics in order to shorten healing time and excretion of the worm.

### EXAMPLE 8

### Treatment of a Plasmodium Blood Infection Using Intravenous or Intraarterial Injection

A subject diagnosed with malaria due to any of the four species of Plasmodium is treated with the composition of the described embodimentby injection delivered by an intravenous drip bag or an intraarterial syringe. The intravenous or intraarterial injection contains either an effective amount of the composition disclosed herein by itself, or a mixture of an effective dose of the composition and an effective dose of the appropriate chemotherapeutic agent, such as chloroquine phosphate, sulfonamides, and primethamine. The composition is dispersed into the blood stream and the liver by injection wherein the protozoan lives in order to eliminate it from the patient and reduce inflammation. Intravenous or intraarterial injection is given hourly to reduce discomfort in the subject. Application of the solutions will expedite the riddance of Plasmodium from the human host.

### EXAMPLE 9

### Treatment of Diarrhea by Oral Administration of a Capsule

A subject suffering from diarrhea due to any number of pathogenic geni, speci, or strain of Escherichia coli, Proteus mirabilis, Salmonella enteritidis, Klebsiella Yersinia, Shigella, Serratia, Candida, Giardia intestinalis, Cryptosporidium, Vibrio cholera, Campylobacter, Ascaris or the like responsible for gastrointestinal distress in the form of diarrhea or the like is treated with a capsule containing the ROM inhibitory compound NADPH oxidase inhibitor diphenlyeneiodonium in a concentration of 0.8% by weight of formulation in addition to administration of the appropriate chemotherapeutic compound, such as an aminoglycoside, chloramphenicol, trimethoprimsulfa-methoxazole, doxycycline, or a fluoroquinolone. The treatment consists of oral intake of three to ten capsules per day for a period of seven to thirty days (depending the causative agent of the infection). The administration of diphenlyeneiodonium is effective in accelerating the treatment of diarrhea in the subject.

### EXAMPLE 10

### Treatment of Sepsis Using Intravenous or Intraarterial Injection

A subject diagnosed with sepsis is treated with the composition of the described embodiment by injection delivered by an intravenous drip bag or an intraarterial syringe. The intravenous or intraarterial injection contains either an effective amount of the composition disclosed herein by itself, or a mixture of an effective dose of the composition and an effective dose of the appropriate chemotherapeutic agent specific to the organism responsible for the sepsis. The composition is dispersed into the blood stream by injection wherein the sepsis organism dwells. Intravenous or intraarterial injection is given hourly to reduce discomfort in the subject. Application of the solutions will expedite the riddance the causative agent of sepsis from the subject.

### EXAMPLE 111

### Treatment of Dental Carries Using a Toothpaste and Mouthwash

A subject suffering from dental carries due to Streptococcus mutans is treated with a toothpaste and a mouthwash containing the ROM inhibitory compound NADPH oxidase inhibitor diphenlyeneiodonium in a concentration of 0.05% by weight of formulation. The treatment consists of five tooth brushings with this toothpaste and mouthwash applications per day for a period of seven days. The administration of diphenlyeneiodonium is effective in treating the dental carrier of the subject.

What is claimed is:

1. A method for inhibiting and reducing enzymatically produced ROM-mediated oxidative damage to a patient's skin or mucosal membranes comprising the step of topically delivering an effective dose of a ROM production and release inhibitory compound in a pharmaceutically acceptable carrier to a subject suffering from ROM-mediated oxidative damage to said patient's are of infection.

2. The method of claim 1, wherein said ROM-mediated oxidative damage to said patient area of infection is a bacterial disease selected from the group comprising Streptococcus, Staphylococcus, members of the family of Enterobacteriaceae, Helicobacter, Neisseria, Chlamydia, Mycobacterium, Treponema, Pseudomonas, Haemophilus, Mycoplasma, Clostridium, Actinobacillus, Rickettsia, Legionella, Listeria, and Leptospira.

3. The method of claim 1, wherein said ROM-mediated oxidative damage to said patient's skin or mucosal membranes is a fungal disease selected from the group comprising Tinea, Candida, Histoplasma, Sporothrix, Blastomycoides, Cryptococcus, Aspergillus, and Malassezia.

4. The method of claim 1, wherein said ROM-mediated oxidative damage to said patient's skin or mucosal membranes is a helminth disease selected from the group comprising Ascaris, Diphyllobothrium, Gnathostoma, Wuchereria, Brugia, Onchocerca, Loa Loa, and Mansonella.

5. The method of claim 1, wherein said ROM-mediated oxidative damage to said patient's skin or mucosal membranes is a protozoan disease selected from the group comprising Plasmodium, Giardia, Trichomonas, Toxoplasma, and Leishmania.

6. The method of claim 1, wherein said ROM production and release inhibitory compound is selected from the group consisting of histamine, histamine dihydrochloride, histamine diphosphate, other histamine salts, esters, prodrugs, H₂ receptor agonists, serotonin, and 5HT agonists.

7. The method of claim 1, wherein said ROM production and release inhibitory compound is a compound that promotes the release of endogenous histamine stores.

8. The method of claim 7, wherein said compound that promotes the release of endogenous histamine stores is selected from the group consisting of IL-3, retinoic acid, 9-cis-retinoic acid, all-trans-retinoic acid, and allergens.

9. A method for making a composition for topically delivering a compound that inhibits the production and release of enzymatically produced ROMs comprising:
providing a pharmaceutically acceptable carrier and histamine in a concentration effective to treat a ROM mediated damage to skin caused by a microbial infection; and
forming a composition containing the pharmaceutically acceptable carrier and said compound that inhibits the production and release of enzymatically produced ROMs.

10. The method of claim 9, wherein said compound is selected from the group consisting of histamine, histamine dihydrochloride, histamine diphosphate, other histamine salts, esters, prodrugs, H₂ receptor agonists, serotonin, and 5HT agonists.

11. The method of claim 9, wherein said compound is a compound that promotes the release of endogenous histamine stores.

12. The method of claim 9, wherein said pharmaceutically acceptable carrier is a lozenge, mouthwash, toothpaste, cosmetic, transdermal patch, intravenous injection, intraarterial injection, suppository, enema, eye drop, ointment, lotion, surgical implant, controlled release mechanism, soap, pill, capsule, vapor, spray, or wound dressing.

13. The method of claim 9, wherein the method of treatment is for helminth, yeast, fungal, protozoan, or other parasitic infectious diseases which cause inflammation.

14. A method for treating a microbial infection comprising the steps of:
diagnosing a patient with a microbial infection;
administering to that patient an effective amount of the appropriate chemotherapy; and
administering to that patient a compound effective to inhibit the production or release of intracellular hydrogen peroxide selected form the group consisting of histamine, other H₂ receptor agonists, and serotonin.

15. The method of claim 14, wherein the administration of said appropriate chemotherapy and said compound effective to inhibit the production or release of intracellular hydrogen peroxide is performed simultaneously.

16. The method of claim 14, wherein the administration of said appropriate chemotherapy is performed within 1 hour of the administration of said compound effective to inhibit the production or release of intracellular hydrogen peroxide.

17. The method of claim 14, wherein said compound effective to inhibit the production or release of intracellular hydrogen peroxide is administered in a dose of from about 0.1 to about 10 mg/day.

18. The method of claim 14, wherein said compound effective to inhibit the production or release of intracellular hydrogen peroxide is administered alone.

19. The method of claim 14, wherein said compound effective to inhibit the production or release of intracellular hydrogen peroxide is administered in combination with an effective dose of the appropriate chemotherapeutic.

20. The method of claim 14, wherein said compound is a compound that promotes the release of endogenous histamine stores selected from the group consisting of IL-3, retinoic acid, 9-cis-retinoic acid, all-trans-retinoic acid, and allergens.
